(19) United States
(12) Patent Application Publication
(10) Pub. No.: US 2026/0368786 A1
(43) Pub. Date: Nov. 19, 2026

(54) METHOD AND SYSTEM FOR INCONTINENCE PRODUCT ASSESSMENT

(71) Applicant: Essity Hygiene and Health Aktiebolag, Goteborg (SE)

(72) Inventors: Nicole Huige, Zeist (NL); Linda Rönn, GÖTEBORG (SE); Natalie Harris, GÖTEBORG (SE)

(21) Appl. No.: 19/135,320

(22) PCT Filed: Dec. 6, 2022
(86) PCT No.: PCT/EP2022/084631

§ 371 Date: Jun. 3, 2025

(51) Int. Cl. G06Q 30/0282; G16H 10/60

(52) U.S. Cl. CPC G06Q 30/0282; G16H 10/60

(57) ABSTRACT

Method and system for performing computer-based comparison of processes of use of sets of incontinence products. A processor of a computer device such as an evaluation server is configured to receive, for each wearer from a plurality of wearers in a care system, first assessment time series data regarding a first process of use of a first set of one or more incontinence products worn by the wearer, second assessment time series data regarding a second process of use of a second set of one or more incontinent products worn the wearer, and a wearer profile record. The processor is further configured to generate values of a plurality of evaluation parameters indicating a comparison of the first process of use and the second process of use, based on processing the first and second assessment time series data, and the wearer profile record for each wearer from the plurality of wearers.

### CROSS REFERENCE TO RELATED APPLICATIONS

THIS APPLICATION IS A NATIONAL STAGE APPLICATION OF PCT/EP2022/084631, FILED DEC. 6, 2022, THE CONTENTS OF WHICH ARE INCORPORATED BY REFERENCE IN THEIR ENTIRETY HEREIN.

### TECHNICAL FIELD

The disclosure relates generally to an evaluation server and an interactive assessment tool for computer assessment and representation that facilitates a process of use of incontinence products.

### BACKGROUND

Hygiene products such as, e.g., incontinence products, are widely used worldwide by various groups of persons who suffer from incontinence, i.e. involuntary and uncontrolled excretion of bladder or bowel contents, or have troubles with independently using a toilet. For example, it is relatively common that elderly, disabled, and bedridden persons in hospitals, nursing homes and similar institutions suffer from incontinence and consequently may not always anticipate or control the need for toilet visits. For this reason, it is common for such persons to use absorbent products such as incontinence pads, pants, napkins, diapers, etc. Such persons typically need assistance from nurses or other caretakers for manually checking whether the incontinence product needs changing.

As the population worldwide is generally aging, nursing homes, hospitals, and other care systems may become overwhelmed with labor and costs required to attend to persons with incontinence problems. More specifically, it may be challenging to determine which incontinence products to use, in a manner that allows reducing costs and manual efforts required to operate the care system.

Conventionally, a decision to order a certain product is based on properties of the products and, mainly, its cost. A product manufacturer or distributor may provide physical samples of a product for use in a care system, and residents may be offered to wear the products. Assessment of new or modified products, if performed at all, is typically manual and subjective. Moreover, selection of products may be made without considering an effect that a product change may have on the entire care system.

Thus, there remains a need for improved methods for appropriate managing of selection and use of incontinence products.

### SUMMARY

The object of embodiments of the present disclosure is to address the above need in improved techniques for assessment of a process of use of incontinence, or containment products of various types.

Embodiments of the present disclosure provide a technique that addresses the above need. In particular, a computer assessment tool or platform is provided for computer assessment and representation of a process of use of incontinence products. A system including an evaluation server and the assessment tool in accordance with aspects of the present disclosure allows deriving values of a plurality of evaluation parameters related to computer-generated evaluation of a process of use of a certain set of one or more incontinence products. The evaluation server and the assessment tool perform computer-generated comparison of processes of use of incontinence products of different sets. In this way, results of operation of the evaluation server and the assessment tool allow indicating a value, to a care system, of selecting an incontinence product of a particular set, versus incontinence product(s) of one or more other sets.

According to an aspect of the disclosure, a computer-implemented method is provided that is performed by at least one processor of an evaluation server configured to:
for each wearer from a plurality of wearers in a care system,
   receive, during a first assessment time period, first assessment time series data regarding a first process of use of a first set of one or more incontinence products worn by the wearer;
   receive, during a second assessment time period, second assessment time series data regarding a second process of use of a second set of one or more incontinent products worn the wearer; and
   receive a wearer profile record being created for the wearer, the wearer profile record indicating a profile group from the plurality of profile groups to which the wearer is assigned and metadata related to the wearer, wherein:
      the first assessment time series data includes assessment values of first assessment parameters related to the use of the first set of one or more incontinence products by the wearer, and the second assessment time series data includes assessment values of second assessment parameters related to the use of the second set of incontinence products by the wearer;
      the first process of use includes a plurality of first steps and the second process of use includes a plurality of second steps; and
      for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generate values of a plurality of evaluation parameters indicating a comparison of the first process of use and the second process of use, based on processing the first assessment time series data, the second assessment time series data, and the wearer profile record for each wearer from the plurality of wearers.

According to an aspect of the disclosure, the object is achieved by an inventive concept disclosed herein. Hereby, a technical effect includes determining values of evaluation parameters related to the process of use of one or more incontinence products of a certain set that is evaluated for adoption by a care system for a certain wearer. Values of evaluation parameters determined regarding a first process of use of a first set of one or more incontinence products are compared to values of evaluation parameters determined regarding a second process of use of a second set of one or more incontinence products. In this way, the present systems and methods allow using computer-based assessment and evaluation to generate values of evaluation parameters that provide a computer representation of an effect or impact on the care system and individual wearers of a change from a certain set of incontinence products to another set of incontinence products, wherein the change may be to and from different sets of products for different wearers in the care system. In this way, it becomes possible to automatically assess an impact of a change between sets of incontinence products in a care system, which advantageously allows improving wearer's well-being and saving costs and time related to operating the care system. Also, environmental impact of the operation of the care system may be reduced.

According to an aspect of the disclosure, a computer-implemented method is provided that is performed by at least one processor of an evaluation server configured to:

In certain examples, the values of the plurality of evaluation parameters indicate a comparison between a first plurality of evaluation parameters related to computer-generated evaluation of the first process of use of the first set, and a second plurality of evaluation parameters related to computer-generated evaluation of the second process of use of the second set.

In certain examples, the values of the plurality of evaluation parameters indicate a ratio between the first plurality of evaluation parameters and the second plurality of evaluation parameters.

In certain examples, the method further includes, after receiving the first assessment time series data, receiving sensor data acquired from a test incontinence product, the test incontinence product being used by the wearer during an information collecting time period, and the test incontinence product including a wireless sensor configured to measure voiding activity of the wearer using the test incontinence product as the sensor data including measured test voiding data, and configured to send the measured test voiding data to the evaluation server.

In certain examples, the second set of one or more incontinent products may be selected based on the sensor data acquired from the test incontinence product.

In certain examples, the first assessment time series data and the second assessment time series data are received via an assessment user interface of an assessment tool.

In certain examples, the first assessment time period and the second assessment time period may have the same duration of time. In certain examples, the first assessment time period and the second assessment time period may have different durations of time.

In certain examples, the method may further include instructing rendering a computer representation of one or more values of the plurality of evaluation parameters on a user interface. In certain examples, the user interface may include a filter-based user interface including user-selectable filter fields for selecting at least one or more profile groups from the plurality of profile groups. In certain examples, the filter-based user interface may be configured to present, in response to receiving user input via the user-selectable filter fields, a computer representation of one or more values of the plurality of evaluation parameters corresponding to the selected one or more profile groups.

In certain examples, the step of generating a computer representation of one or more values of the plurality of evaluation parameters may include generating a computer representation of one or more values of the plurality of evaluation parameters corresponding to selected two or more profile groups, such as three or more profile groups or all profile groups of the care system.

For example, the evaluation parameters may include one or more, such as at least two of or all of: a total absorption change, skin health, care efficiency (staff of care giver's time); sustainability, and a total economic value change. For example, the evaluation parameters may include at least care efficiency (staff or care giver's time) or total economic value change, including costs for staff or care giver's time.

In certain examples, the computer representation of the one or more values of the plurality of evaluation parameters may include a first icon representing a corresponding evaluation parameter from the plurality of evaluation parameters and a second icon related to the first icon and representing a value of the corresponding evaluation parameter.

In certain examples, the wearer is assigned to the profile group from the plurality of profile groups based at least on a voiding pattern of the wearer, mobility of the wearer, and a cognitive ability of the wearer.

In certain examples, the first assessment time series data includes one or more out of skin assessment, a change of an incontinence product of the first set, feces in the incontinence product of the first set, a toilet visit, a cleansing and protection event, an occurrence of urine leakage, a clothes change event, a linen change event, an application of a day type of the incontinence product of the first set, and an application of a night type of the incontinence product of the first set.

In certain examples, the second assessment time series data includes one or more out of skin assessment, a change of the incontinence product of the second set, feces in the incontinence product of the second set, a toilet visit, a cleansing and protection event, an occurrence of urine leakage, a clothes change event, a linen change event, an application of a day type of the incontinence product of the second set, and an application of a night type of the incontinence product of the second set.

In certain examples, the first assessment parameters related to the use of the first set of one or more incontinence products by the wearer and the second assessment parameters related to the use of the second set of one or more incontinence products by the wearer are same parameters.

In certain examples, the method may further include, when the value of at least one of the plurality of evaluation parameters has reached or exceeded a threshold, automatically transmitting, to an external computer, a request for a certain amount of incontinence products of the second set to be delivered to the care system.

In certain examples, the method may further include automatically generating a recommendation for the care system regarding the second process of use of the second set of one or more incontinence products. In certain examples, the recommendation may include a recommendation for a modification to at least one step from the plurality of second steps of the second process of use of the second set of one or more incontinence products. In certain examples, the recommendation may include a recommendation for a modification to a configuration of an incontinence product of the second set.

In certain examples, the method may further include generating an indication to the care system, the indication recommending that the care system uses incontinence products of the second set.

In certain examples, the method may further include automatically generating a recommendation for the care system regarding the second process of use of the second set of one or more incontinence products. In certain examples, the recommendation may include a recommendation for a modification to at least one step from the plurality of second steps of the second process of use of the second ser of one or more incontinence products. In certain examples, the recommendation may include a recommendation for a modification to a configuration of an incontinence product of the second set. In certain examples, the recommendation for the care system may be generated using the sensor data acquired from the test incontinence product.

According to an aspect of the disclosure, an evaluation server is provided including at least one processor that is configured:
for each wearer from a plurality of wearers in a care system:
   receive, during a first assessment time period, first assessment time series data regarding a first process of use of a first set of one or more incontinence products worn by the wearer;
   receive, during a second assessment time period, second assessment time series data regarding a second process of use of a second set of one or more incontinent products worn the wearer; and
   receive a wearer profile record being created for the wearer, the wearer profile record indicating a profile group from the plurality of profile groups to which the wearer is assigned and metadata related to the wearer, wherein:
      the first assessment time series data includes assessment values of first assessment parameters related to the use of the first set of one or more incontinence products by the wearer, and the second assessment time series data includes assessment values of second assessment parameters related to the use of the second set of incontinence products by the wearer;
      the first process of use includes a plurality of first steps and the second process of use includes a plurality of second steps; and
      for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generate values of a plurality of evaluation parameters indicating a comparison of the first process of use and the second process of use, based on processing the first assessment time series data, the second assessment time series data, and the wearer profile record for each wearer from the plurality of wearers.

In certain examples, the at least one processor of the evaluation server is configured to perform a method in accordance with embodiments of the present disclosure.

In certain examples, the first assessment time series data and the second assessment time series data are received from the computer device.

According to an aspect of the disclosure, a system including a computer device and an evaluation server is provided. The evaluation server includes at least one processor that is configured to:
for each wearer from a plurality of wearers in a care system,
   receive, during a first assessment time period, first assessment time series data regarding a first process of use of a first set of one or more incontinence products worn by the wearer;
   receive, during a second assessment time period, second assessment time series data regarding a second process of use of a second set of one or more incontinent products worn the wearer;
   receive a wearer profile record being created for the wearer, the wearer profile record indicating a profile group from the plurality of profile groups to which the wearer is assigned and metadata related to the wearer, wherein:
      the first assessment time series data includes assessment values of first assessment parameters related to the use of the first set of one or more incontinence products by the wearer, and the second assessment time series data includes assessment values of second assessment parameters related to the use of the second set of incontinence products by the wearer;
      the first process of use includes a plurality of first steps and the second process of use includes a plurality of second steps; and
      for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generate values of a plurality of evaluation parameters indicating a comparison of the first process of use and the second process of use, based on processing the first assessment time series data, the second assessment time series data, and the wearer profile record for each wearer from the plurality of wearers.

In certain examples, in the system, the at least one processor of the evaluation server is configured to perform a method in accordance with embodiments of the present disclosure.

According to an aspect of the disclosure, a computer program product including instructions, which, when executed on at least one processor, cause the at least one processor to carry out a method in accordance with embodiments of the present disclosure.

According to an aspect of the disclosure, a computer-readable storage medium is provided. The computer-readable storage medium has stored thereon a computer program product including instructions which, when executed on at least one processor, cause the at least one processor to carry out a method in accordance with embodiments of the present disclosure.

Additional features and advantages are disclosed in the following description, claims, and drawings. Furthermore, additional advantages will be readily apparent from the present disclosure to those skilled in the art or recognized by practicing the disclosure as described herein. There are also disclosed herein control units, computer program products, and computer-readable media associated with the above discussed technical effects and corresponding advantages.

### BRIEF DESCRIPTION OF THE DRAWINGS

With reference to the appended drawings, below follows a more detailed description of aspects of the disclosure cited as examples.

FIG. 1 is a block diagram illustrating an example of architecture of a computer system in which a method in accordance with aspects of the present disclosure may be implemented.

FIGS. 2A and 2B are flowcharts illustrating an example of a computer-implemented method in accordance with aspects of the present disclosure.

FIG. 3 is a diagram illustrating communication of devices in the computer system of FIG. 1.

FIG. 4 is a graph illustrating a method of selecting an incontinence product of a second set, in accordance with aspects of the present disclosure.

FIG. 5 is a flowchart illustrating an example of a computer-implemented method in accordance with aspects of the present disclosure.

FIGS. 6A, 6B, 6C, 6D, and 6E are block diagrams illustrating examples of an assessment tool of the computer system of FIG. 1, in accordance with aspects of the present disclosure.

FIGS. 7A and 7B are block diagrams illustrating examples of a filter-based user interface, in accordance with aspects of the present disclosure.

FIG. 8 is a snapshot of an example of a filter-based user interface in accordance with aspects of the present disclosure.

FIG. 9 is a snapshot of another example of a filter-based user interface in accordance with aspects of the present disclosure.

FIG. 10 is a schematic diagram of an exemplary computer device for implementing examples disclosed herein, in accordance with aspects of the present disclosure.

FIG. 11 is a schematic diagram of an exemplary computer server for implementing examples disclosed herein, in accordance with aspects of the present disclosure.

### DETAILED DESCRIPTION

Various incontinence products have been developed for comfort and dignity of elderly persons and persons with disabilities, such as, e.g., long-term care residents. At the same time, selection of products of an appropriate type, as well as determining a number of products required, e.g., in a certain care facility or system, remains to be a challenge. Selecting a product for wearers is often performed manually, without considering person's characteristics, and without determining an effect that a selection of a certain product, or a type of the products, may have on wearers and on the overall operation of the care system. Although systems and digital tools exist that computerize managing persons in care systems, there remains a need for improved computational methods for acquiring analyzing, and using data related to use of incontinence products in a care system or another setting.

Accordingly, the object of embodiments of the present disclosure is to provide a system and method for facilitating acquisition and processing of data related to a process of use of an incontinence product of a certain type, and automatic decision-making regarding a selection of the incontinence product.

The object is achieved by providing a technology-implemented interactive tool, referred to herein as an assessment platform or tool implemented in a system including a computer device and an evaluation server in communication with the computer device and other computer devices. The assessment platform or tool is executed by the computer device. Processing circuitry of the computer device and processing circuitry of the evaluation server are configured to perform methods in accordance with aspects of the present disclosure, as described in detail below.

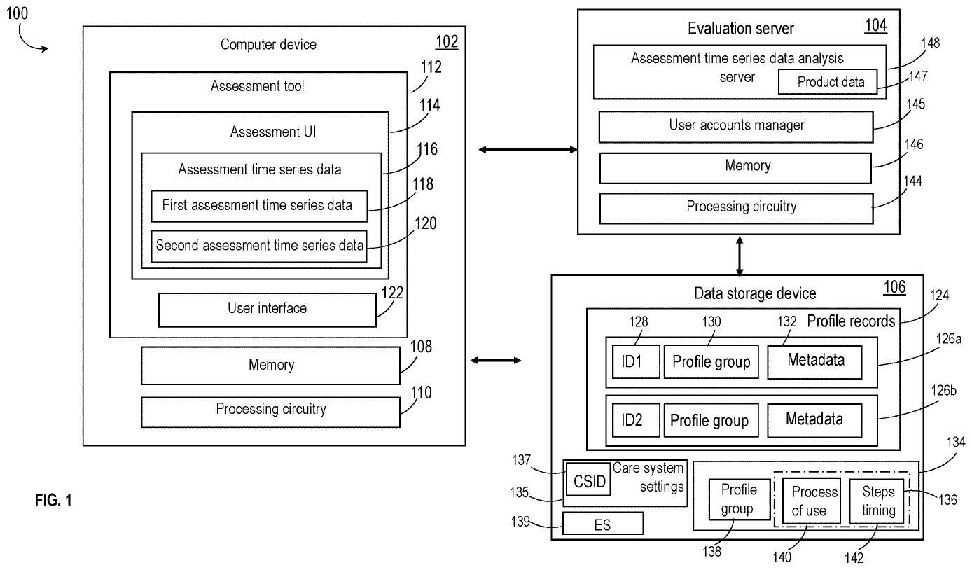

FIG. 1 depicts a computer system 100 according to an example embodiment of the present disclosure. The computer system 100 includes a computer system or device 102 and an evaluation server 104 which communicate via a network such as a wireless communications network. The computer system 100 also includes a data storage device 106, which is accessible to the computer device 102 and the evaluation server 104.

The computer device 102, such as a personal computer, a smartphone, a tablet, a personal digital assistant (PDA), or any other user electronic device, may be associated with a user such as a care system and/or a care giver. The computer system or device 102 may encompass more than one, e.g., two or more, computer devices associated with and controlled by a care system such as a person or a group of persons in the care system. A plurality of wearers of incontinence products may be associated with the care system. In some cases, the computer device 102 may be a specialized computer device configured for use by staff in a care system such as a nursing home, hospice, or other facility. It should be noted however that the computer device 102 may be any type of a computer device as aspects of the present disclosure are not limited in this respect.

The evaluation server 104 may be a machine such as a computer server in communication with multiple user computer devices. The computer devices may be associated with different care systems, each having one or more computer devices associated therewith. The computer device(s) of care systems may be registered with the evaluation server 104, e.g., a care system or a another user such as person may have an account created with the evaluation server 104 for that user. The user is defined as a user of services provided by the evaluation server 104, such as services provided by an assessment platform or tool. As shown in FIG. 1, the evaluation server 104 may include a user accounts manager 145 that stores account information for each registered user, such as a care system or person, and manages the accounts and communications with the users. The evaluation server 104 may acquire data from multiple users and it may provide processing results, such as values of a plurality of evaluation parameters related to computer-generated evaluation of a process of use of a certain set of one or more incontinence products worn by the wearer, e.g. a second process of use of a second set of one or more incontinence products worn by the wearer, computed based on data acquired from multiple care systems, as discussed in more detail below. The evaluation server 104 may be a physical server. In some embodiments, the evaluation server 104 may be a cloud server running in a cloud computing environment, i.e. it may be virtualized and its resources may be available to users remotely over a wireless communications network.

As shown in FIG. 1, the computer device 102 has a memory 108 storing computer-executable instructions, and processing circuitry 110 operatively connected to the memory 108 and that is configured to execute the computer-executable instructions that cause the processing circuitry 110 to perform methods in accordance with aspects of the present disclosure. The memory 108 may include one or more storage media such as physical devices, including for example, a hard-drive, flash memory, permanent memory such as read-only memory (“ROM”), semi-permanent memory such as random access memory (“RAM”), any other suitable type of storage component, or any combination thereof. The processing circuitry 110 may control writing data to and reading data from the memory 108. To perform any of the functionality described herein, the processing circuitry 110 may execute one or more processor-executable instructions stored in one or more non-transitory computer-readable storage media e.g., the memory 108, which may serve as non-transitory computer-readable storage media storing processor-executable instructions for execution by the processing circuitry 110.

The computer device 102 may include an evaluation and assessment tool 112, referred to herein as an assessment tool, which may be, e.g., a platform or an application (or an app) installed on the computer device 102 or accessed via the computer device 102. For example, the assessment tool 112 may be downloaded from the evaluation server 104, or from another computing device or server, and installed for execution on the computer device 102. In some cases, the assessment tool 112 may be run on more than one physical computer device, such that it may be accessed, e.g., in the care system, via two or more computer devices. The evaluation server 104 may be a cloud server and the assessment tool 112 may be run on the server 104 and thus accessible via a wireless communications network.

The assessment tool 112 may present an assessment User Interface (UI) 114 on the computer device 102 or on a display associated with the computer device 102. The assessment UI 114 is a graphics user interface that is configured to present fields and/or other UI elements for receiving user input data regarding a process of use of an incontinence product worn by a wearer. The assessment UI 114 is thus used to acquire the user input data which may be processed by the assessment tool 112 and sent, by the assessment tool 112, to the evaluation server 104.

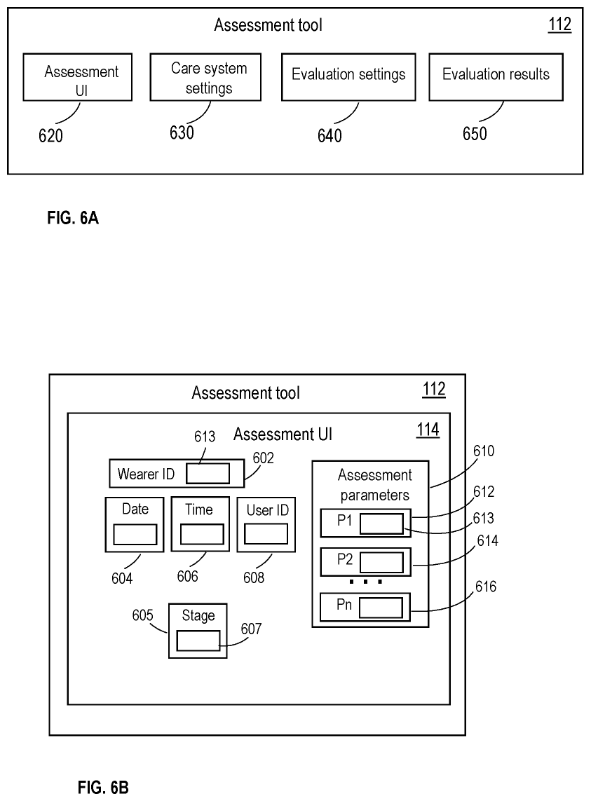

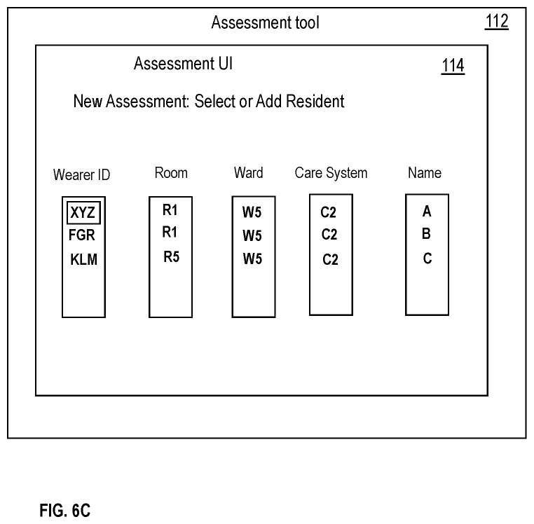

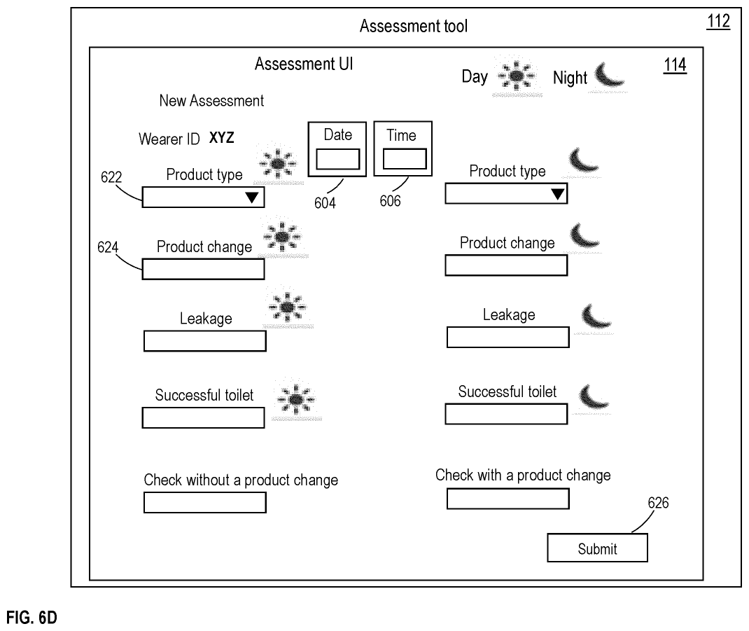

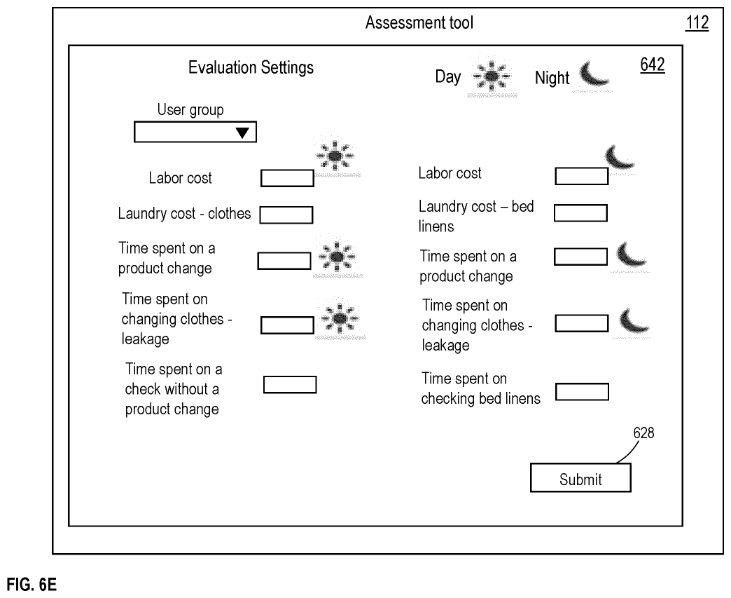

In some examples, the assessment tool 112 may present, in addition to the assessment UI 114, a UI for receiving care system settings, a UI for receiving evaluation settings, and a UI for presenting evaluation results. Other UIs may be presented additionally or alternatively. The different UIs may be accessed via the assessment tool 112, as discussed in more detail below in connection with FIG. 6A. Access to some of the UIs of the assessment tool 112 may be restricted. For example, in a care system, personnel may encompass persons having different levels or tiers of access to information presented in the UIs rendered by the assessment tool 112. The information presented on one or more of the UIs may be allowed to be modified only by persons having a certain level of access. For example, the assessment UI 114 may be configured to receive user input data from a care giver or personnel of a similar type. Management personnel may have access to the assessment UI 114, and may be able to edit information in the assessment UI. At the same time, access to the UI for receiving care system settings, the UI for receiving evaluation settings, and the UI for presenting evaluation results may be limited to management personnel, which may also have different tiers of access. For example, one type of management personnel may be allowed to access the UI for receiving care system settings, whereas another type of management personnel may be allowed to access the UI for presenting evaluation results.

As shown schematically in FIG. 1, the assessment UI 114 may be used to acquire assessment time series data 116, such as, e.g., first assessment time series data 118 and second assessment time series data 120, which may be acquired regarding a process of use of respective sets of incontinence products. The system and method in accordance with aspects of the present disclosure provide a platform for computational analysis of data acquired regarding processes of use of sets of incontinence products, such that the sets of incontinence products may be compared to one another. A set of incontinence products, also referred to herein as an incontinence product set, may include one or more products of one or more types. For example, an incontinence product set may include products of a day type i.e. intended to be worn during the day, products of a night type i.e. intended to be worn during the night, and products defined otherwise. A set of incontinence products encompasses incontinence products that are worn by a certain wearer during an assessment time period and regarding which an assessment is being performed. A set of incontinence products, including one or more products, is associated with a process of use of the one or more products in the set.

For a certain wearer, assessment time series data may be acquired for a set of incontinence products that are different from another set of incontinence products that the wearer had previously worn and for which data for comparison exists. In examples herein, the first assessment time series data may be a first set of one or more incontinence products, the first set being associated with a first process of use including a first plurality of steps. The first set may be a set of incontinence products that was previously worn by the wearer. The second assessment time series data may be a second set of one or more incontinence products, the second set being associated with a second process of use including a second plurality of steps, wherein the second set is a set of incontinence products being assessed during a current assessment period and being compared to the first set of incontinence products. As used herein, “previously worn” may mean that the wearer had been wearing the products from the first set before the current assessment period, or at any other time prior to the current assessment. In other words, the first set of incontinence products may be a set of any types of inconsistence products for which assessment time series data, referred to herein as a first assessment time series data, has been acquired. Furthermore, in some examples, the first assessment time series data is acquired immediately before the second assessment time series data is acquired, i.e. the current assessment period directly follows an assessment period during which a plurality of evaluation parameters were generated regarding the first process of use of the first set of incontinence products. It should be appreciated that the first assessment time series data may be acquired regarding a first process of use of a first set of incontinence products at any time in relation to acquisition of the second assessment time series data, including only during an assessment time period rather than being in use in a care system. Regardless of the timing of acquisition of the first assessment time series data, this data is considered to be a baseline or base data, whereas the second assessment time series data is considered to be an effect data which is compared to the base data, as discussed in more detail below.

As an example, the first assessment time series data 118 may be acquired regarding the first set of one or more incontinence products worn by a wearer and the second assessment time series data 120 may be acquired regarding the second set of one or more incontinence products worn by the wearer when it is desired to assess whether a change from the first set of incontinence products to the second set of incontinence products is predicted to be beneficial to the wearer and/or to the care system. For a certain wearer, both the first assessment time series data 118 and the second assessment time series data 120 may be acquired.

Furthermore, during the assessment time period, processes of use of different sets of incontinence products may be analyzed, including sets that are different among at least some of the wearers, such that effect of changes from and to different products is evaluated for different wearers.

The second set of incontinence products includes one or more products that are being evaluated using the computational tools in accordance with embodiments of the present disclosure, and the second process of use of which is being assessed as compared to a first process of use of the first set of incontinence products. The system and methods in accordance with embodiments of the present disclosure allow generating user interface data such as e.g., graphical user interface data and representations that indicate advantages to the wearer and to the care system of the use of the second set of incontinence products relative to the use of the first set of incontinence products. The “use,” unless specified otherwise, as related to a set of incontinence products, may be referred to one or both the act of wearing the one or more incontinence products by a wearer in a care system, and procedures performed by the care system in relation to the incontinence products, such as order, purchase, administration to wearer(s), and disposal of the incontinence product.

The second set of incontinence products may be associated with the second process of use which is applied to administer the one or more incontinence products of the second set to a wearer. The first and second processes of use may include similar or the same steps, such that respective data acquired regarding these processes are comparable, which thus allows comparison of the first process and the second process. The system and method in accordance with aspects of the present disclosure provide a technique that automatically generates values of a plurality of evaluation parameters indicating a comparison of the first process of use of the first set of incontinence products worn by the wearer and the second process of use of the second set of incontinence products worn by the wearer, as discussed in more detail below.

In some embodiments, the first assessment time series data 118 is time series data that is acquired in connection with a (first) process of use of one or more incontinence products of a first set worn by a wearer from a plurality of wearers in a care system. The second assessment time series data 120 may be time series data that is acquired in connection with a (second) process of use of one or more incontinence products of a second set worn by the wearer. In some examples herein, the second assessment time series data 120 is referred to as the assessment time series data.

The computer device 102 may acquire information from the data storage device 106 shown in FIG. 1. Although the data storage device 106, such as a memory device, is shown as a separate device, it may in some cases be part of the computer device 102. In some cases, the data storage device 106 may be part of the evaluation server 104. The data storage device 106 may be a distributed memory device. Regardless of its specific implementation, the data storage device 106 may store profile records 124, a profile group table 134, care system settings 135, and evaluation settings 139. Each profile record in the profile records 124 may include a wearer profile record stored in the data storage device 106 and created for the wearer from the plurality of wearer, e.g., wearers in a care system.

As shown in FIG. 1, where two wearer profile records 126a, 126b are shown by way of example, each profile record, such as a first profile record 126a discussed herein as an example of profile records, includes multiple fields storing an identifier (e.g., ID1) 128 of the wearer for which the profile was created, a profile group 130 from the plurality of profile groups to which the wearer is assigned, and the metadata 132 related to the wearer. Each of the identifier 128, the profile group 130, and the metadata 132 may be stored in respective different fields of the profile record 126a. Furthermore, some of the fields, e.g., the field storing the metadata, may encompass more than one field. The respective fields of a second profile record 126b, including an identifier ID2 of another wearer, may be similar, and are not labeled in FIG. 1.

In some cases, the identifier 128, such as, e.g., ID1 shown in FIG. 1, of a wearer of an incontinence product may be a unique, de-personalized identifier that identifies the respective (first) profile record 126a and does not include any features that would allow identifying the wearer. In some cases, the identifier may be a unique, personalized identifier that may be removed when the data in the profile record 126a is acquired from the data storage device 106 and used in an analysis. The profile group 130 stored in the first record 126a indicates a profile group from a plurality of profile groups to which the wearer has been assigned. The wearer may be assigned to the profile group from the plurality of profile groups based at least on a voiding pattern of the wearer, mobility of the wearer, and a cognitive ability of the wearer. For example, in some embodiments, six profile groups, also referred to as care groups or care need groups, are defined for wearers having respective different ability regarding use of incontinence products.

In some embodiments, a specific graphical user interface may be used to acquire user input data regarding the voiding pattern of the wearer, mobility of the wearer, the cognitive ability of the wearer, and other features of the wearer. The graphical user interface may be presented by a profile group assessment tool, which may be installed and run on the computer device 102 or on another computer device. In some examples, the profile group assessment tool is part of the assessment tool 112. In some examples, the profile group assessment tool may be separate from the assessment tool 112 and may be executed independently of the assessment tool 112.

Metadata in a wearer profile record, e.g., the metadata 132 in the profile record 126a created to store data related to the respective wearer, includes wearer's name, address, existing conditions, history, and other attributes or features. The metadata 132 may also include information on a location of the wearer, e.g., a care system, a ward in the care system, a room, in the ward, etc. Information on a carer who has cared or is caring for the wearer may also be included in the metadata 132. The information on the care system may include, e.g., an identifier of the care system which may be used to link this information in the metadata 132 to care system settings in the care system settings 135, identified by the care system identifier 137.

Furthermore, the metadata 132 may also include information on incontinence products previously and/or currently worn by the wearer. The incontinence products may belong to one or more sets of incontinence products. The information on these incontinence products may be defined in the product data storage 147.

The profile records 124 in the data storage device 106 may be linked, via a profile group field, e.g., the field storing the profile group 130 of the profile 126a, to a profile group table 134 that stores profile group records for each of the plurality of profile groups. Thus, as shown in FIG. 1 illustrating one profile group record 136 for a profile group 138, the profile group record 136 stores, for a step from the plurality of steps of a process of use of an incontinence, an estimated duration of time required to perform that step. The profile group table 134 indicates a working time required to perform different tasks per profile group. As an example, as shown in FIG. 1, the profile group record 136 stores an indication 140 of a process of use of a set of incontinence products and an indication 142, denoted as “Steps timing” in FIG. 1, of an estimated duration of time required to perform each step of a plurality of steps of the process use indicated by the indication 140. For each profile group from the plurality of profile groups, a task or step from the plurality of tasks or steps is associated with an estimated duration of time required to perform the step. The profile group record 136 for the profile group 138 thus stores three-dimensional or greater-dimensional data including, for each process of use and for each step of that process, an estimated duration of time required to perform that step. Thus, a working time for a certain task, such as e.g. change of an incontinence product, change of clothes, change of linens, etc., for a certain care or profile group, may be specified. The information stored in the respective profile group record 136 for each profile group 138 may be the same for different care systems. In some examples, the information stored in the respective profile group record 136 for each profile group 138 may vary among different care system, e.g. depending on specifics of completion of tasks in the care system, a type and amount of equipment used in the care home where the equipment is for facilitating different kinds of work steps, and/or based on other factors.

Access to the information stored in the profile group record 136 may be restricted in some examples. For example, only personnel with specific access permissions may be able to access the information stored in the profile group record 136. In some examples, the information stored in the profile group record 136 may be provided by a manufacturer of the incontinence products. In a care system, the personnel with specific access permissions may be able to modify the information in the profile group record 136.

The evaluation server 104 includes processing circuitry or or processor device 144 and memory 146 operatively coupled to the processing circuitry 144 and storing computer-executable instructions that, when executed by the processing circuitry 144, perform the methods in accordance with aspects of the present disclosure. In some cases, the evaluation server 104 includes an assessment time series data analysis server 148 that is configured to generate or determine values of a plurality of evaluation parameters related to computer-generated evaluation of a process of use of a set of incontinence products. The assessment time series data analysis server 148 may be configured to derive values of the plurality of evaluation parameters which may be e.g., in the form of a comparison between a plurality of evaluation parameters related to a process of use of a first set of one or more incontinence products worn by the wearer, also referred to herein as a first plurality of evaluation parameters related to a first process of use of the first set of incontinence products; and a plurality of evaluation parameters related to a process of use of the second set of incontinence products worn by the wearer, also referred to as a second plurality of evaluation parameters related to a second process of use of the second set of incontinence products. The values of the plurality of evaluation parameters may indicate a ratio between the first plurality of evaluation parameters and the second plurality of evaluation parameters.

In some examples, to determine values of evaluation parameters, the assessment time series data analysis server 148 may use product data regarding the incontinence products and a context data such as care system settings related to the care system. The product data, which may be stored in the product data storage 147, may define properties of the product, a process of manufacturing of the products, a process of disposing of the products after being worn, etc. Non-limiting examples of the product data regarding the incontinence products include a CO₂ footprint from producing the products, a weight of disposed material after using the product i.e. if the product is disposable, an amount of non-renewable material in the product, and other types of data. The product data may have numerical values and/or other types of values.

The product data stored in the product data storage 147 may be provided by the incontinence product manufacturer and in some cases the information the product data storage 147 may be updatable. Thus, as the assessment time series data analysis server 148 receives, along with the assessment time series data, an indication regarding which set of incontinence products is used, the assessment time series data analysis server 148 may access the data storage 147 storing the product data related to the set of incontinence products. In some cases, different product data may be associated with one or more incontinence products in a given set—e.g., day and night products of the same set may have different values of a CO₂ footprint from producing the products, and/or values of other types of product data may differ among incontinence products in the same set. The data product storage 147, shown in FIG. 1 as part of the evaluation server 104, may be located e.g. in memory 146 of the evaluation server 104 or in another memory device, and may be a table or other data structure that is updatable. For example, information in the data storage may be updated if e.g. the manufacturing process of a product is altered such that its CO₂ footprint is reduced, e.g. if the manufacturing switches from less-sustainable energy sources to e.g. wind or solar power. The data product storage 147, although shown in FIG. 1 as part of the evaluation server 104, may be located in the data storage device 106. Information in the data product storage 147 may be accessible through the assessment tool 112.

The care system settings 135, such as context data regarding a certain care system, may include one or more of a staff working hour, cost for laundry, CO₂ impact of laundry, cost of waste disposal, CO₂ impact of waste disposal e.g. transport to landfill, incineration, etc., as related to the process of use of sets of incontinence products. As shown in FIG. 1, the care system settings 135 may include a care system identifier (CSID) 137 identifying the care system for which data on care system settings is stored as the care system settings 135. The care system settings 135 may store settings for one or more care systems. Also, for each care system e.g., identified by the care system identifier 137, information on smaller divisions of that care system may also be stored such as, e.g., information on a ward in the care system, a room in the ward, etc. As the care system may have any structure, for each care system, related type of data may be stored. It should be noted that, in some cases, a care system may belong to a group of one or more or a plurality of care systems that are related to one another, e.g. managed by a certain entity such as a private or public entity. In such cases, the care system identifier 137 may additionally indicate with which care system of one or more care systems the wearer is associated.

In FIG. 1, the care system settings 135 are shown to be stored in the data storage 106. However, the care system settings 135 may be stored in least in part in the computer device 102, or in another computer device accessible to the care system. The data or information in the care system settings 135 may be updatable by a care system—e.g., the care system may update e.g. the cost for staff working hours and/or the costs for laundry as agreements with staff/laundry suppliers are updated. These values may be determined by each care system, and they may be relatively straightforward to acquire. In some examples, the information in the care system settings 135 may be accessible and updatable through a care system settings UI.

In some examples, the care system settings 135 may include, at least in part, the profile group table 134 storing the profile group record 136 for the profile group 138.

Some of the data included in the care system settings 135, such as e.g. the CO₂ impact of laundry or of waste disposal, may be less straightforward to determine. Thus, the relevant values may be estimated at different geographical or other types of scales. For example, a CO₂ impact of laundry may be estimated generally, as “in Europe”, or “in a specific country in Europe,” or “in a specific city in Europe”. Accordingly, at least some of the data in the care system settings 135 may depend on a geographical location of the care system. Thus, the assessment time series data analysis server 148 may obtain different data in the care system settings 135 depending on the location of the care system such as e.g. region, country, city, etc. The relevant values are updated as more related information becomes available.

Accordingly, in examples herein, the product data and the care system settings data may be separately acquired and updated. Furthermore, the system in accordance with examples herein allows making relevant comparisons which are customized to relevant markets or geographic areas. For example, what is considered a better sustainability choice for one market or for one care system may not be a better choice for another market or care system.

In some examples, the assessment time series data analysis server 148 may use a suitable computational approach, e.g., an artificial intelligence approach, to determine the values of the evaluation parameters. In some embodiments, assessment time series data analysis server 148 may execute a machine learning model that includes a logistic regression classifier. In some embodiments, the machine learning or deep learning model can be one or more out of a decision tree, an ensemble such as e.g., bagging, boosting, or random forest, gradient boosting machine, linear regression, Naïve Bayes, or a neural network.

In some embodiments, the assessment time series data analysis server 148 may use one or more of: supervised learning, e.g., using logistic regression, using back propagation neural networks, using random forests, decision trees, etc.; unsupervised learning, e.g., using an Apriori algorithm, using K-means clustering); semi-supervised learning; a deep learning algorithm, e.g., neural networks, a convolutional neural network method, a recurrent neural network method; reinforcement learning, e.g., using a Q-learning algorithm, using temporal difference learning; a regression algorithm, e.g., ordinary least squares, logistic regression, stepwise regression, multivariate adaptive regression splines, locally estimated scatterplot smoothing, etc.; an instance-based method, e.g., k-nearest neighbor, learning vector quantization, self-organizing map, etc.; a regularization method, e.g., ridge regression, least absolute shrinkage and selection operator, elastic net, etc.; a decision tree learning method, e.g., classification and regression tree, chi-squared automatic interaction detection, decision stump, random forest, multivariate adaptive regression splines, gradient boosting machines, etc.; a Bayesian method, e.g., naïve Bayes, averaged one-dependence estimators, Bayesian belief network, etc.; a kernel method, e.g., a support vector machine, a radial basis function, a linear discriminate analysis, etc.; a clustering method, e.g., k-means clustering, expectation maximization, etc.; an ensemble method, e.g., boosting, boostrapped aggregation, AdaBoost, stacked generalization, gradient boosting machine method, random forest method, etc., and/or any suitable artificial intelligence approach.

The computer device 102 receives, e.g., from the assessment tool 112, the assessment time series data 116 including assessment values of assessment parameters related to the use of a certain set of incontinence products worn by the wearer. The computer device 102, in response to receiving the assessment time series, sends the assessment time series data, e.g., via a network connection such as a secure network connection, to the evaluation server 104. The computer device 102 also acquires, from the data storage device 106, the wearer profile record stored in the data storage device 106, and the computer device 102 sends the wearer profile record to the evaluation server 104. For example, the assessment time series data analysis server 148 at the evaluation server 104 may receive the assessment time series data and the wearer profile record. It should be noted that, in some cases, the metadata from the wearer profile record may not be sent to the evaluation server 104, or only part of the data in the metadata from the wearer profile record may be sent to the evaluation server 104.

The assessment time series data 116 may include the first assessment time series data 118 and the second assessment time series data 120, which may be sent to the evaluation server 104 simultaneously or at different times. In some implementations, the first assessment time series data 118 is sent to the evaluation server 104 before the second assessment time series data 120. For example, the first assessment time series data 118 may be sent to the evaluation server 104 as a result of computer evaluation of a first process of use of a first set of one or more incontinence products, and the second assessment time series data 120 may be sent to the evaluation server 104 as a result of computer evaluation of a second process of use of a second set of one or more incontinence products. The first assessment time series data 118 and the second assessment time series data 120 may be stored in the evaluation server 104 once they are received by the evaluation server 104. In some embodiments, the first assessment time series data 118 may be stored in the evaluation server 104 and, once the second assessment time series data 120 is received by the evaluation server 104, the server 104 analyzes both the first assessment time series data 118 and the second assessment time series data 120, as discussed in more detail below.

In some examples, the computer device 102 may also acquire, from the data storage device 106, information on care system settings from the care system settings 135 stored in the data storage device 106. The care system settings may be acquired for a particular care system, e.g., as identified by the care system identifier 137 in the care system settings 135. The computer device 102 sends the acquired care system settings to the evaluation server 104. Furthermore, in some examples, the care system settings may be acquired from the data storage device 106 by the evaluation server 104. For example, in some implementations, the care system identifier is provided by the computer device 102 to the evaluation server 104 e.g. as part of the metadata from the wearer profile record, and the evaluation server 104 may acquire the required information from the care system settings 135 based on the care system identifier shown as the care system identifier 137 in FIG. 1.

The evaluation server 104 and/or its component, e.g., the assessment time series data analysis server 148, analyzes the assessment time series data and data in the wearer profile record, and additionally other relevant data, and determines values of a plurality of evaluation parameters related to computer-generated evaluation of the process of use of a certain set of one or more incontinence products worn by the wearer. As used herein, “worn by the wearer” means worn during a certain period of time, such as an assessment time period. Also, the set of incontinence products is considered to be worn when one or more types of incontinence product belonging to the set are worn by a wearer during the assessment time period. The act of wearing refers to an individual incontinence product that is worn by as wearer at a certain time period within the assessment time period. As a non-limiting example, a wearer may wear three day type products from the set and two night type products from the set per a 24-hour period, wherein the assessment time period may include several days.

The evaluation server 104 may send the values of the plurality of evaluation parameters to the computer device 102 that may, in response to receiving the values of the plurality of evaluation parameters, render a user interface (UI), also referred to herein as a filter-based UI, 122 including user-selectable filter fields for selecting one or more profile groups from the plurality of profile groups and/or one or more care systems from the one or more care systems. The filter-based UI 122, e.g., an evaluation results UI, which is discussed in more detail below, is configured to present, in response to receiving user input via the user-selectable filter fields, a computer representation of one or more values of the plurality of evaluation parameters corresponding to the selected one or more profile groups and/or one or more care systems. The filter-based UI 122 may be rendered by the assessment tool 112 on the same computer devices as the assessment UI 114 or on a different computer device.

It should be appreciated that the computer device 102, the data storage device 106, and the evaluation server 104 of the system 100 of FIG. 1 may include various other components implemented in hardware, software, firmware, or any combination thereof, which are not shown in FIG. 1. For example, the computer device 102, the evaluation server 104, and a computer device through which the data storage device 106 is accessed, which may be one or both of the computer device 102 and the evaluation server, may each include a communication interface including a transmitter, a receiver, a transceiver and/or one or more antennas. Furthermore, one or more of the computer devices in which embodiments of the present disclosure may be implemented may be e.g. terminals, wireless communications terminals, internet of things (NB-IoT) devices, device to device (D2D) terminals, or nodes such as e.g. a smartphone, laptop, mobile phone, or mobile tablet.

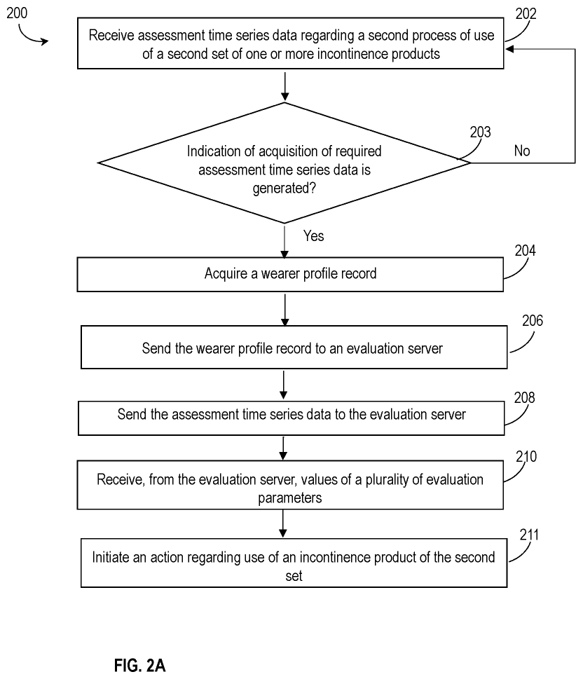

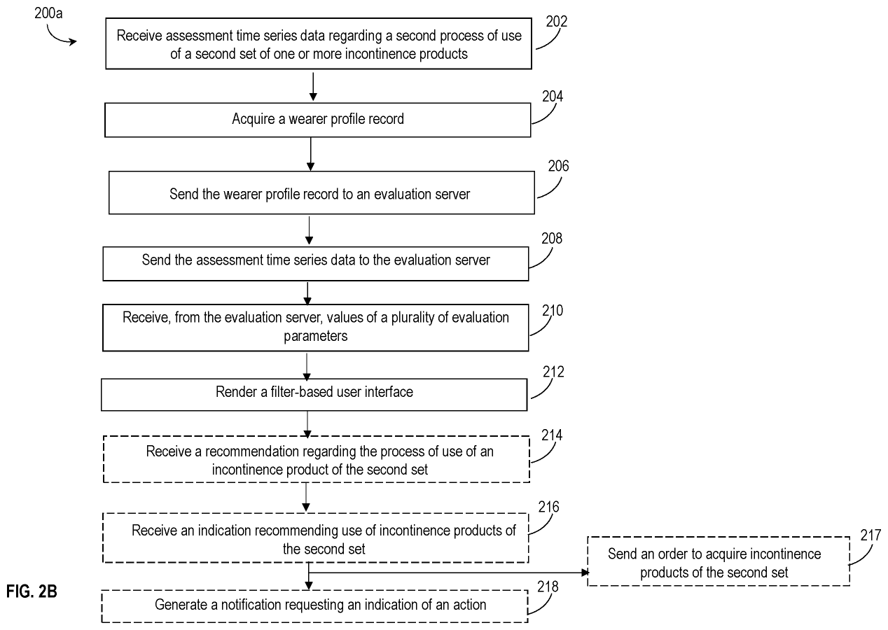

FIGS. 2A and 2B illustrate an example embodiment of a computer-implemented method or process 200, 200a including executing at least one processor, such as processing circuitry, of a computer device 102 to perform steps or actions of the process. It should be noted that, unless indicated otherwise, actions at blocks of FIGS. 2A and 2B can be performed in any suitable order which can be different from the illustrated order. Optional actions are shown using dashed lines.

The process 200 of FIG. 2A may be performed by a computer system having a configuration as shown, e.g., for system 100 of FIG. 1. Thus, for example, the processing circuitry 110 of the computer device 102 of a care system may execute the process 200. The process 200 may begin, for example, when an application or platform, e.g., the assessment tool 112 e.g. executed by the processing circuitry 110, is initiated. The process 200 may be executed for each wearer from a plurality of wearers in the care system from may belong to one or more care systems. The plurality of wearers may not necessarily encompass every person associated with the care system and wearing an incontinence product, but a group of wearers may be selected to assess a process of use of a certain set of one or more incontinence products. It should be noted that the first and second sets may be different for difference wearers, such that a change regarding two different sets may be assessed for each wearer, in an individualized manner. In other words, for each wearer, a different pair of the first and second sets may be assessed. In certain examples, for some wearers, the same pairs of the first and second sets may be assessed.

At block 202, the process 200 includes, for each wearer from a plurality of wearers in a care system from one or more care systems, receiving, via the computer device, during an assessment time period, assessment time series data regarding a second process of use of a second set of one or more incontinence products worn by the wearer. The assessment time series data includes assessment values of assessment parameters related to the use of the second set of one or more incontinence products worn by the wearer, the second process of use of the second set of one or more incontinence products includes a plurality of second steps, and, for each profile group from a plurality of profile groups, a step from the plurality of second steps is associated with an estimated duration of time required to perform the step. As mentioned above, the second set of incontinence products is defined herein as one or more incontinence products associated with a second process of use of the second set, which is different from a first set of one or more incontinence product associated with a first process of use of the first set. As used herein, the second set is considered to be different from the first set when either at least one of the incontinence products in the second set is different from at least one of the incontinence product of the first set, and/or when at least one step of the second process of use of the second set is different from at least one step of a first process of use of the first set. In other words, there is a difference among the incontinence products of the first and second sets, and/or there is a difference among the first process of use and the second process of use. In some examples, the second set of incontinence products includes at least one incontinence product of a type that has not yet been worn by a particular wearer, such that this product may be considered new for the wearer.

Furthermore, as mentioned above, during the assessment time period, processes of use of different sets of incontinence products may be analyzed. In some examples, the second set of one or more incontinence products and the first set of one or more incontinence products are different among at least some wearers from the plurality of wearers. For example, for a certain wearer, the first assessment time series data may be acquired regarding a process of use of a set A and the second assessment time series data may be acquired regarding a process of use of a set B; for another wearer, the first assessment time series data may be acquired regarding a process of use of a set C and the second assessment time series data may be acquired regarding a process of use of a set D; for another wearer, the first assessment time series data may be acquired regarding a process of use of the set A and the second assessment time series data may be acquired regarding a process of use of the set C; and, for yet another wearer, the first assessment time series data may be acquired regarding a process of use of the set B and the second assessment time series data may be acquired regarding a process of use of the set A. The effect of the change from one set of incontinence products to another set, for multiple wearers in the care system, may thus be assessed. Thus, aspects of the present disclosure allow assessing, in an efficient and robust manner, the effect on operation and cost-efficiency of the entire care system of individualized changes in incontinence products worn by different wearers in the care system.

The assessment time series data, also referred to herein as the second assessment time series data, includes assessment values of assessment parameters related to the use of incontinence products of the second set worn by the wearer during the assessment time period. The second set of incontinence products may include one type, and typically more than one type of incontinence products, and (second) assessment time series data is acquired as a product from the set is being worn by the wearer, and between the product wearing events—e.g., a toilet visit, a laundry change event, a skin treatment application event, etc. The second process of use of the second set of incontinence products includes a plurality of second steps. As used herein, the second process of use of the second set of incontinence products may be defined as the process spanning the entire assessment time period, and the second process of use may include steps other than those directly related to the act of wearing an incontinence product from the second set by the wearer. For example, the second process of use may include skin assessment, a change of the incontinence product, a toilet visit, a cleansing and protection event, a clothes change event, a linen change event, an application of an incontinence product of a day type from the second set, an application of an incontinence product of a night type from the second set, and other steps. For each profile group from a plurality of profile groups, a step from the plurality of second steps, of the second process of use of the second set of incontinence products, is associated with an estimated duration of time required to perform the step. The assessment time series data may include assessment values of assessment parameters some of which may coincide with the steps of the second process. For example, the assessment values may include a number of occurrences of skin assessment, a number of occurrences of a change of the incontinence product, a number of toilet visits which may be a number of successful toilet visits, a number of occurrences of a cleansing and protection event, a number of occurrences of a clothes change event, a number of occurrences of a linen change event, a number of occurrences of an application of an incontinence product of a day type from the second set, and a number of occurrences of an application of an incontinence product of a night type from the second set. The assessment time series data may also include values e.g. a number of occurrences of such assessment parameters as clothes laundry, linens laundry, a leakage, etc. As used herein, the term “leakage” means a leakage from an incontinence product to the outside of the incontinence product, i.e. an undesired leakage of urine or feces not properly contained by the incontinence product.

As discussed above, the assessment time series data may be acquired via a graphical user interface of an assessment tool executed on the computer device, e.g., assessment UI 114 of assessment tool 112 of FIG. 1. Examples of the assessment UI 114 are shown in FIGS. 6B, 6C, and 6D. FIG. 6A illustrates how the assessment UI 114, as well as other UIs, may be accessed. In FIG. 6B, the assessment UI 114 includes a wearer ID field 602 including an interactive field 613 configured to receive input identifying the wearer. The wearer ID field 602 can be configured to receive data that is the same as the identifier stored in a profile record for the wearer, e.g., in the profile records storage 124 in data storage device 106 of FIG. 1. The profile record for the wearer may be created before or after the assessment time series data is acquired in connection with the use of the incontinence products of the second set worn by the wearer. In some cases, the wearer ID field 602 in the assessment UI 114 is configured to receive wearer identification data that is different from the identifier used to identify the profile record stored in connection with data on that wearer.

As shown in FIG. 6B, the assessment UI 114 includes a date field 604, time field 606, and a user ID field 608, each including a respective interactive field configured to receive respective data. In some implementations, some or all of these fields may be filled in automatically.

The user ID field 608 is configured to receive data identifying the user of the assessment tool 112 such as a carer or caregiver, or any person administering an incontinence product to the wearer. In some implementations, the date field 604 and time field 606 may not be present, since the time and date may be recorded automatically by the processor executing the assessment UI 114. For example, the date field 604 and time field 606 may be automatically selected based on a process of use of the incontinence product and/or based on pre-defined time points within the assessment time period. Furthermore, the respective interactive field of at least the time field 606 may be present and filled in automatically, and the automatic entry may be adjustable by the user, such as e.g. a carer or care giver. For example, if a product check is performed that has not been planned i.e, that is not part of a pre-defined routine, the day/time for such event may be recorded manually.

The assessment UI 114 may further include an assessment parameters field or panel 610 that includes fields for receiving assessment values of the assessment parameters related to the use of the incontinence product of the second set worn by the wearer. In the example of FIG. 6B, fields for a first parameter, P1, 612, a second parameter, P2, 614, and an nth parameter, Pn, 616 are shown to be included in the assessment UI 114. An interactive field 613 for the first parameter P1 612 is labeled. It should be noted that three parameter fields are shown as an example only, as values of any number of assessment parameters may be acquired via the assessment UI 114. The values may be of a suitable type, depending on the parameter. For example, the values may be textual, numerical, two-valued (e.g., binary), multi-valued, etc., or any combination thereof.

As shown in FIG. 6B, the assessment UI 114 may also include a stage field 605 including a feature 607 such as, e.g., a pulldown menu or another digital object that can receive an indication of a selection between stages of acquisition of assessment time series data-base or baseline measurements of assessment time series data and effect measurements of assessment time series data. As used herein, the baseline measurements are defined as acquisition of assessment time series data regarding a process of use of a certain set of one or more incontinence products that is, for purposes of comparison, considered to be a baseline set to which a comparison is to be made. Furthermore, as used herein, the effect measurements are defined as acquisition of assessment time series data regarding a process of use of another set of one or more incontinence products that is compared to the baseline set. The effects of the change from a base or baseline set of incontinence products, referred to herein as the first set of incontinence products, to another set of incontinence product, referred to herein as the second set of incontinence products, are thus computationally determined, which may be done, e.g., before a decision is made to switch to another, second, set of incontinence products. The decision may be made automatically.

For example, in use, the effect measurements may be acquired regarding a new (e.g., newly developed, or new to a wearer i.e. not previously worn by the wearer, or new to a care system, or otherwise defined as new) set of incontinence products that is different from a first set of incontinence products. For a certain wearer, the first set may include incontinence products previously worn by the wearer. It should be noted that the second set may also include incontinence product(s) previously worn by the wearer, but assessment time series data may not have been acquired regarding a process of use of such products. Moreover, as mentioned above, in some cases, the second set of incontinence products may be different from the first set of incontinence products in the sense that at least one step in the second process of use of the second set is different from at least one step in the first process of use of the first set. In such cases, both the second set and the first set may include the same one or more types of incontinence products, and only the respective processes of their use may differ. However, for the purposes of the present disclosure, such sets are still referred to as the second and first sets.

Accordingly, comparison between sets including any types of incontinence products may be performed, whereas one set is considered to be a baseline set and another set is compared to the baseline set. The incontinence products in the sets being compared may be comparable products such that, e.g., their processes of use may be the same or similar, and the same or similar assessment time series data may thus be acquired as part of product sets comparison.

In examples herein, the baseline incontinence product set may be a first set of incontinence products, and baseline measurements may include first assessment time series data regarding a first process of use of the first set of incontinence products worn by a wearer from a plurality of wearers in a care system from one or more care systems. A set of incontinence products compared to the baseline incontinence product set may be a second set of incontinence products, and effect measurements may include second assessment time series data regarding a second process of use of the second set of incontinence products worn by the wearer.

The assessment parameters acquired over the assessment time period may be selected so as to simplify the process of acquisition of their respective values at time points of the assessment time period. Accordingly, in some embodiments, the assessment parameters may be selected such that their values may all be binary, and thus input indicating a value of an assessment parameter may be a simple check of a checkbox or field, an entry of a “yes” or “no” value, or another choice between two options. In this way, the assessment tool is configured to receive the values of the assessment parameters in a simplified format, which also advantageously facilitates a process of data entry by a user of the assessment tool in the care system. Caregivers assigned the task of caring for wearers needing incontinence products may already be overwhelmed by their daily tasks, and providing the tool in accordance with aspects of the present disclosure, that allows them to continue their work without significant disruption, may significantly improve the process of adoption of a new incontinence product by the care system.

Non-limiting examples of the assessment parameters include one or more out of skin assessment, a change of an incontinence product of the second set, feces in an incontinence product of the second set, a toilet visit such as a successful toilet visit, a cleansing and protection event, an occurrence of urine leakage, a clothes change event, a linen change event, an application of a day type of an incontinence product of the second set, or an application of a night type of an incontinence product of the second set. The clothes change event and the linen change event are events that may be caused by a leakage event. Any other assessment parameters may be used additionally or alternatively. The assessment tool may receive user input indicating a value of one or more of the assessment parameters, at a time point during the assessment time period. For example, based on the observation by the caregiver, user input may be received indicating whether or not a clothes change event took place at a certain time point during the assessment time period. At least some of the assessment parameters may correspond to steps from the plurality of second steps of the second process of use of the second set of incontinence products. The second process is performed, e.g., by a caregiver that is assisting a wearer with incontinence products of the second set, as well as with other tasks related to wearing the incontinence products. Each step of the plurality of second steps may be associated with an estimated duration of time required to perform that step. Thus, for example, for a clothes change event, a time that is required for the clothes change is acquired from a corresponding profile group record in a profile group table such as e.g. a profile group table 134 shown in FIG. 1.

It should be noted that the time required for a certain step or task is an estimated time, which may be different depending on a profile group to which the specific wearer is assigned. The value of the estimated time may be adjustable, e.g., by a manufacturer of relevant one or more incontinence products, and/or by a care system e.g. by a specific person in the care system.

Furthermore, some of the assessment parameters, e.g., an occurrence of urine leakage, an occurrence of solid waste leakage, or an occurrence of (any type of) leakage, etc. may not directly correspond to steps of the second process as they are based on observations by the caregiver rather than being steps or tasks performed by the caregiver. At the same time, an occurrence of a leakage is typically determined as a result of performance of a task such as an incontinence product check, which may be e.g. a product check with a product change, a product check without a product change, a product check with a linens change, a product check without a linens change, or an occurrence of another type of event, or an observation.

The second set of one or more incontinence products may include a night type product configured to be worn at night and a date type product configured to be worn during the day. Both the day and night type products may be referred to as incontinence products of the second set since they possess similar features, and both or one of them may be different, in at least some features, from an incontinence product of the first set.

The assessment time series data may be acquired in connection with use of incontinence products of the second set of a day type, or a night type, or another type e.g. a product not specifically designed to be worn during a specific time; and values of one or two respective assessment parameters may be acquired, e.g., via the assessment UI 114 of FIG. 1. The assessment UI 114 may be configured to indicate a specific product included in the second set, or in any other set being assessed.

The assessment UI 114 may include other fields or other digital objects that are not shown in FIG. 6B. FIGS. 6C and 6D illustrate additional examples of the assessment UI 114. FIG. 6A illustrates that different UIs may be accessed via the assessment tool 112. In the example of FIG. 6A, user-selectable features such as icons are shown that are selectable to provide access to respective UIs. Thus, the assessment tool 112 includes an assessment UI icon 620 selectable to provide access to the assessment UI such as e.g. the assessment tool 114, a care system settings UI icon 630 selectable to provide access to a care system settings UI, an evaluation settings UI icon 640 selectable to provide access to an evaluation settings UI, and an evaluation results UI icon 650 selectable to provide access to an evaluation results UI. In some implementations, instead of separate evaluation settings UI and care system settings UI, one UI, e.g., a care system settings UI, may be provided.

The access to the different UIs that can be accessed via the assessment tool 112 may be restricted to certain persons or entities. For example, the assessment UI may be accessible to caregivers, whereas the care system settings UI, the evaluation settings UI, and the evaluation results UI may be accessible to management personnel. In some cases, different levels of access are provided to the information presented on and modifiable via the care system settings UI, the evaluation settings UI, and the evaluation results UI, such that, e.g. different persons or groups of persons associated with a care system may have access to these UIs.

FIG. 6C illustrates an example of a UI such as the assessment UI 114 for a new assessment which may include selecting or adding a wearer, shown as “New Assessment: Select or Add Resident”. The assessment UI 114 in FIG. 6C includes user-selectable panels Wearer ID, Room, Ward, Care System, and Name, with the specific order of the panels being shown by way of example only. Also, the entries in these panels are shown in FIG. 6C as an example only. Data for three wearers is shown as an example. Also, FIG. 6C shows that a wearer having a wearer ID “XYZ” is selected such that assessment of a process of use of a certain set of one or more incontinence products may proceed in connection with the selected wearer. For example, in response to receiving user input indicating a selection of a specific wearer, via that wearer's ID, another UI or panel may overlay the UI shown in FIG. 6C. Data on a wearer may be added using the shown UI, such that assessment for that wearer may proceed.

FIG. 6D illustrates an example that is a continuation of the example of FIG. 6C. In FIG. 6D, after the wearer ID “XYZ” is selected through the assessment UI 114 as shown in FIG. 6C, information on a certain set of incontinence products, such as assessment values of assessment parameters, may be received regarding the wearer identified by the wearer ID “XYZ”. In this example, the assessment parameters include a product type for each of a day and night type product, a product change event for each of a day and night type products, a leakage event for each of a day and night type products, a successful toilet visit during day and during night, a check without a product change, and a check with a product change. A value of the assessment parameter may be received via a corresponding field, e.g., a field 622 for a product type, a field 624 for a product change event, etc. The assessment UI 114 of FIG. 6D may also include date field 604 and time field 606 similar to such fields shown in FIG. 6B. In addition, a submit button 626 is shown in FIG. 6D which may be activated, e.g., via a user input, to submit the information received via the assessment UI 114. The submitted information may be sent to a server, such as, e.g., the evaluation server 104 shown in FIG. 1.

Furthermore, FIG. 6E illustrates an example of an evaluation settings UI 642 that may be accessed via the assessment tool 112, e.g., the evaluation settings UI icon 640 shown in FIG. 6A. The evaluation settings UI 642 may receive user input regarding values of certain values per a user group. As shown, non-limiting examples of the evaluation settings include labor cost for each of a day and night type products, cost of clothes laundry, cost of bed linens laundry, a time spent on a product change for each of a day and night type products, a time spent on changing clothes due to leakage for each of a day and night type products, a time spent on a check without a product change, and a time spent on checking bed linens. A submit button 628 is shown in FIG. 6E, which may be activated, e.g., via a user input, to submit the information received via the evaluation settings UI 642. The submitted information may be sent to a server, such as, e.g., the evaluation server 104 shown in FIG. 1.

Data including values of the parameters may be acquired by the assessment UI 114 from the user or from another person having access to the assessment UI 114 during a time when assessment time series data is acquired. The assessment UI 114 may be presented on a touchscreen display. In some implementations, the assessment UI 114 can be a voice user interface (VUI) configured to receive voice and/or speech commands. In some examples, the assessment UI 114 may be configured to receive any type of user input, including a combination of textual, visual, and graphics input. Furthermore, the assessment UI 114 may be configured to issue voice instructions to the user regarding the assessment process, as the assessment time series data is acquired. Thus, the assessment UI 114 may lead the user, who may be engaging in manually handling the incontinence product (e.g., changing it) and assisting the wearer, through a touch-free or hands-free process of entering data in connection with the process of use of the incontinence product of the second set worn by the wearer.

It should be noted that the features shown in FIGS. 6A, 6B, 6C, 6D, 6E, 7A, 7B, 8, and 9 are examples only, as additional features may be displayed, and/or some features may be omitted. Also, specific positions of the features such as various graphical features, elements, fields, buttons, etc., which may be interactive, are shown as examples, for illustration purposes, and not to limit aspects of the present disclosure.

Referring back to FIG. 2A, the assessment time series data may be acquired for a certain time period such as the assessment time period, which may depend on specifics of incontinence products of the second set, a number of wearers in the care system, specifics of the care system, a type of a desired result, and on other factors, including a combination of various factors. For example, the assessment time series data may be acquired during the assessment time period of, for example, from 5 to 7 days or from 5 to 10 days or from 2 to 5 days, or from 3 to 7 days, etc. The assessment time period may have any other duration. Furthermore, the assessment time series data may be acquired for each wearer from a plurality of wearers in a care system. A number of the plurality of wearers for which the assessment time series data may be acquired may less than the entirety of the wearers associated with the care system. For example, the assessment time series data may be acquired for a selected group of wearers.

Thus, as shown in FIG. 2A, it may be determined, at decision block 203, whether an indication of acquisition of the required assessment time series data is generated, wherein the indication indicates that a sufficient amount of assessment time series data is received and/or the certain time period is expired. The sufficient amount of the assessment time series data may mean that the assessment time series data is acquired for all wearers from a plurality of wearers in the care system, and that the assessment time period has expired. The assessment time series data may be acquired as multiple data points at respective time points during the assessment time period, and, in some cases, if certain data is considered missing, a user may be instructed to input further data. For example, additional time points may be added for data collection from the user. The duration of time during which the assessment time series data is acquired may be an adjustable parameter which may be set, e.g., based on user input acquired in connection with setting options presented by the assessment tool.

The indication may be received from the evaluation server, or the indication may be generated by the computer device, e.g., by the assessment tool installed on the computer device. The indication may be rendered on the computer device, or on a display associated with the computer device, e.g., on the assessment UI 114. The indication may be in a textual, audio, gesture-based, tactile, or any other format, and it may be indicating that the process of acquisition of the assessment time series data is to be completed. The indication may be explicit. Additionally or alternatively, in some cases, the indication may be indirect such that the user interface through which the assessment time series data is acquired is disabled and a message is displayed informing the user accordingly.

Responsive to the determination at block 203 that the indication of acquisition of the required assessment time series data is generated, the process 200 may continue to block 204. If it is determined at block 203 that the indication of acquisition of the required assessment time series data is not generated, e.g., the required number of data points in the assessment time series data has not been yet acquired, the process 200 returns to block 202 to acquire further assessment time series data.

As data points in the assessment time series data are acquired at respective time points, the assessment time series data may be stored in memory of the computer device, e.g., in memory 108 of FIG. 1.

At block 204, the processing circuitry acquires, from a data storage device, a wearer profile record stored in the data storage device, the wearer profile record being created for the wearer from the plurality of wearers, and the wearer profile record indicates a profile group from the plurality of profile groups to which the wearer is assigned and metadata related to the wearer. For example, a wearer profile record stored in the data storage device 106, e.g., in profile records storage 124, may be acquired by the computer device 102 such as by the processing circuitry 110. In some examples, the computer device 102 may store the wearer profile records in memory 108. As shown in FIG. 1, a wearer profile record 126a includes the profile group 130 and metadata 132. The wearer profile record is acquired for each of the wearers in the plurality of wearers for which the assessment time series is acquired.

At block 206, the processing circuitry sends the wearer profile record to an evaluation server, such as evaluation server 104 on FIG. 1. The processing circuitry sends the wearer profile record of each of the wearers in the plurality of wearers the evaluation server. It should be noted that the wearer profile records of the wearers may be acquired from the data storage device prior to receiving the assessment time series data at block 202. The received wearer profile records may be sent to the evaluation server before receiving the assessment time series data. In embodiments, the wearer profile records may be acquired from the data storage device in connection with the receiving of the assessment time series data. In other words, in certain examples, the processing at both or one of the blocks 204 and 206 may be performed before the processing at blocks 202 and 203.

In some examples, the wearer profile records of all of the plurality of wearers may be sent to the evaluation server simultaneously. In some examples, depending on a set-up of the assessment in accordance with examples of the present disclosure, wearer profiles records of different wearers may be sent to the evaluation server at different times.

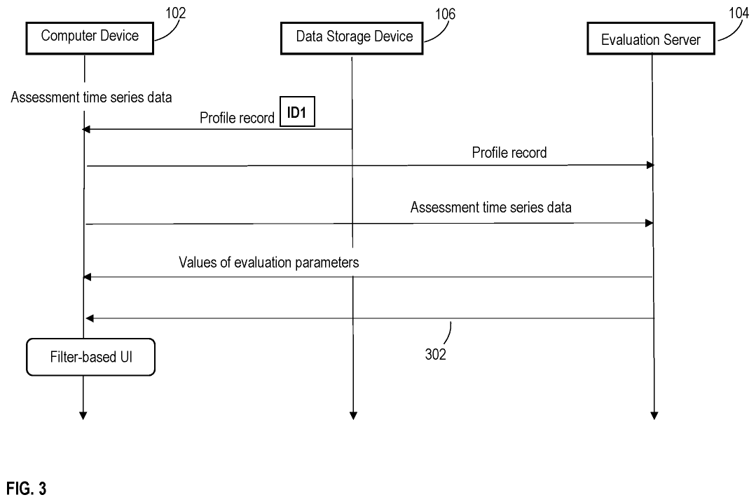

In certain examples, the wearer profile may be identified in the data storage device using a wearer identifier unique to the wearer, and the wearer profile record may be de-identified by dissociating the wearer identifier from the wearer profile record before sending the wearer profile record to the evaluation server. For example, as shown in FIG. 3 discussed below, the wearer profile record acquired by the computer device 102 from the data storage device 106 may include a personalized identifier, shown as ID1, that is unique to the wearer and identifies the wearer and/or the wearer profile record, and the wearer profile record may be de-identified by stripping off this identifier before the wearer profile record is sent to the evaluation server 104.

At block 208, the process 200 includes, in response to receiving the assessment time series data for each of the wearers in the plurality of wearers, sending the assessment time series data, e.g., via a secure network connection, to the evaluation server. As discussed above, in some embodiments, the indication regarding acquisition of the required amount of the assessment time series data may be received. The computer device 102, e.g., the processing circuitry 110, may send the acquired assessment time series data to the evaluation server 104. The assessment time series data analysis server 148, and/or another component of the evaluation server 104, may receive the assessment time series data and data in the wearer profile record (the data being referred to herein as a wearer profile record) of each of the wearers in the plurality of wearers and generate, based on processing of the assessment time series data and the wearer profile record, values of a plurality of evaluation parameters related to computer-generated evaluation of the second process of use of the second set of incontinence products. The assessment time series data analysis server 148 may also access product data such as product data 147 of FIG. 1, care system settings such as stored e.g. in care system settings 135 of FIG. 1, evaluation settings such as stored e.g. in evaluation settings 139 of data storage device 106 of FIG. 1 or in another storage device. The assessment time series data analysis server 148 may further access other data and generate the values of the plurality of evaluation parameters related to computer-generated evaluation of the second process of use of the second set of incontinence products based on processing of the received data.

At block 210, the process 200 includes receiving, from the evaluation server, e.g., via a secure network connection, values of the plurality of evaluation parameters related to computer-generated evaluation of the second process of use of the second set of one or more incontinence products worn by the plurality of wearers, based on processing by the evaluation server of the assessment time series data and the wearer profile record for each wearer from the plurality of wearers. To determine values of the plurality of evaluation parameters, the evaluation server integrates the assessment time series data, related to the second process of use of the second set of one or more incontinence products by the plurality of wearers, with assessment time series data acquired regarding a first process of use of a first set of one or more incontinence products worn by the wearer. As discussed above, baseline measurements may be acquired as assessment time series data acquired regarding a first process of use of the first set of incontinence products, which may be performed in the same way as acquisition of the assessment time series data regarding the second process of use of the second set of incontinence products. Accordingly, the assessment time series data acquired regarding the second process of use of the second set of incontinence products may be referred to as effect measurements. Thus, the evaluation server may determine values of the plurality of evaluation parameters based on processing of both the assessment time series data acquired regarding the first process of use of the first set of incontinence products (also referred to herein as the first assessment time series data) and the assessment time series data acquired regarding the second process of use of the second set of incontinence products (also referred to herein as the second assessment time series data).

The values of the plurality of evaluation parameters determined by the evaluation server thus indicate the computationally assessed effect of the change from the first set of incontinence products to the second set of incontinence products. The effect of the change may be defined in the form of values of evaluation parameters such as, for example, a total absorption change, e.g., per year, month, week, or another time period; skin health, defined, e.g., as a change per year, month, week, or another time period in a number of wearers in need of skin care; care efficiency, defined as, e.g., as a change per year, month, week, or another time period in staff of care giver's time; sustainability, defined, e.g., as a change per year, month, week, or another time period in CO₂ usage by the care system; and a total economic value change. The above are non-limiting examples of the evaluation parameters, and any other number of any other evaluation parameters may be used additionally or alternatively. The values of the evaluation parameters may be defined per a certain time period, or in some cases without reference to a time period.

In an example, the total economic value change may be defined using one or more out of a number of incontinence products needed multiplied by a cost of the incontinence products, a change in staff or care giver time multiplied by an hourly rate of the care giver, a change in amount of laundry required (e.g., in kilograms or kg) multiplied by a cost per kg of laundry, and a change in a number of wearers in need of incontinence associated dermatitis (IAD) treatment multiplied by an average cost of skin treatment. The number of incontinence products of a certain set, e.g., the second set, needed during an assessment time period may be defined as a difference between a number of incontinence products of the first set needed during a time period of the same length. Similarly, the change in a care giver time, amount of laundry washed, and a number of wearers needing IAD treatment may be defined as respective changes relative to a care giver time, amount of laundry washed, and a number of wearers needing IAD treatment recorded for the baseline incontinence product set such as, in embodiments of the present disclosure, one or more incontinence products of the first set.

The process 200 may further include, at block 211, initiating an action regarding use of the incontinence products of the second set in the care system. The action is determined based on the values of the plurality of evaluation parameters received from the evaluation server, and it may be initiated in various way. In some cases, the action may be initiated in response to a trigger received from the evaluation server. The initiation of the action may be, e.g., an indication or a recommendation to the care system regarding the incontinence products of the second set. The indication and/or the recommendation may be presented on a graphical user interface, e.g., on an evaluation results UI of the assessment tool 112, or otherwise displayed on the computer device 102 or on a different computer device. For example, the indication and/or the recommendation may be presented on a filter-based UI, such as, e.g., the filter-based UI 122 of FIG. 1, which may be rendered on the computer device 102 or on a different computer device.

In some examples, as a result of the processing in accordance with a method or process herein, e.g., the process 200 or another process, it may be automatically determined that a change to incontinence products of the second set may not be beneficial for the care system and/or for wearers in the care system. In such examples, an indication and/or the recommendation may be generated and presented that indicates that a switch to the second set from the first set is not recommended based on the computer-implemented evaluation of the assessment time series data and other relevant data. The indication and/or the recommendation may additionally indicate an alternative action, e.g., a suggestion to computationally assess an effect of a change to another, third set of one or more incontinence products. In some cases an alternative action may include a recommendation to continue using incontinence products of the first set in the care system such that one or more wearers would continue wear incontinence products of the first set.

Furthermore, in some examples, it may be automatically determined that a change to incontinence products of some of the assessed seconds set may not be beneficial for the care system and/or for wearers in the care system. As discussed herein, the second set may be different for different wearers such that, for example, for a certain first wearer, the first set of incontinence products may be set A and the second set of incontinence products may be set B; for another, second wearer, the first set of incontinence products may be set C and the second set of incontinence products may be set D; and for another, third wearer, the first set of incontinence products may be set A and the second set of incontinence products may be set C. Each of the first, second, and third wearers may be more than one wearer. The processing in accordance with aspects of the present disclosure may reveal that, for example, while a switch from the set A to set B is beneficial for the first wearer and for the care system and also a switch from the set C to set D is beneficial for the second wearer and for the care system, a switch from the set A to set C for the third wearer is not recommended based on the assessment of the effect of this and other changes. The processing may also generate an indication regarding to which degree a change from the set A to set C for the third wearer would affect a value of one or more evaluation parameter. In this way, the effect of the individualized or personalized changes may be assessed using the methods in accordance with examples herein. The values of the plurality of evaluation parameters may be presented to a user, such as e.g. an operator of the care system or another user, in the manner that allows assessing a possible effect of individualized changes of incontinence products for wearers in the care system.

As an example, an indication may be generated recommending that the care system uses incontinence products of the second set. In some embodiments, in response to the indication, the process 200 may include automatically sending an order to acquire incontinence products of the second set. It should be noted that, since a second set may be different for at least some of the wearers in the care system, an order may be sent regarding incontinence products of different second sets, depending on which second sets were evaluated for different wearers. The order may be sent to a product manufacturer's system/computer, a product storage system/computer, etc. In some embodiments, the initiation of the action may be generation of a recommendation for the care system regarding the process of use of incontinence products of the second set. For example, in some embodiments, the recommendation includes a recommendation for a modification to at least one step from the plurality of second steps of the second process of use of incontinence products of the second set. For example, the recommendation may specify an adjustment to the second process of use of incontinence products of the second set which would advantageously facilitate the use in the care system of the incontinence products of the second set worn by the user. This may relate to reducing a frequency of a change of incontinence products or, for some persons, increasing the frequency of a change which may have such advantages as, e.g., fewer number of linen washes and thus less CO₂ impact, and/or less impact on the wearer's skin. This may ultimately lead to reduced costs of operating the care system where application and handling of incontinence products typically greatly affects the operation costs. Furthermore, because the impact of change of various incontinence products is accessed, the effect or impact of simultaneous change from one set of products to another for separate wearers is assessed. In this way, the effect of individualized changes on the entire care system is assessed in a simplified and robust matter.

Additionally or alternatively, in some embodiments, the recommendation includes a recommendation for a modification to a configuration of incontinence products of the second set.

The process 200 may end at a suitable time, e.g., once the action regarding use of the incontinence product of the second set is initiated.

FIG. 2B illustrates a method or process 200a which is similar to process 200. Actions at blocks 202, 204, 206, 208, and 210 of the process 200a are therefore not discussed in detail herein since they are the same as actions 202, 204, 206, 208, and 210, respectively, of the process 200 of FIG. 2A. The process 200a includes executing at least one processor of a computer device, e.g., computer device 102 of FIG. 1 in communication with evaluation server 104 to perform the steps or actions of the process 200a.

At block 202, the process 200a includes receiving, for each wearer from a plurality of wearers in a care system from one or more care systems, via the computer device, during an assessment time period, assessment time series data regarding a second process of use of a second set of one or more incontinence products worn by the wearer. The assessment time series data includes assessment values of assessment parameters related to the use of the second set of one or more incontinence products worn by the wearer. The second process of use of the second set of one or more incontinence products includes a plurality of second steps. For each profile group from a plurality of profile groups, a step from the plurality of second steps is associated with an estimated duration of time required to perform the step.

The assessment time series data may be received via an assessment user interface of an assessment tool installed on the computer device. For example, assessment time series data may be received via the assessment UI 114 of the assessment tool 112 of FIG. 1.

In some embodiments, the assessment time series data is received as user input from a care giver, and the method further includes authenticating the care giver using a computer identification device. The computer identification device may be worn or carried by the care giver or it may be associated with a device worn by the care giver. The computer identification device may include one or more of a Radio-frequency identification (RFID) tag, a barcode, and a Quick Response (QR) code tag that is configured to identify the care giver. Any other computer identification device may be used. The assessment tool may be configured to automatically detect the computer identification device, for example, when the computer device through which the assessment tool is accessed employs Near Field Communications (NFC) technology to wirelessly communicate with the computer identification device. Once the computer identification device is detected and the care giver is thereby identified, the assessment tool may record an identifier of the care giver. For example, in some implementations, the identifier of the care giver may be automatically entered into the user ID field 608 in the assessment UI 114 of FIG. 1.

Furthermore, in some embodiments, the computer identification device may be detected by a proximity detection device associated with a room in which the wearer is located, such that the authentication is performed automatically as the care giver is located in proximity to the room and the proximity detection device detects the computer identification device. In such embodiments, the proximity detection device may communicate wirelessly with the computer device, which would typically be carried by the care giver, and the proximity detection device may be configured to perform the care giver identification and to provide the identifier of the care giver to the assessment tool.

In some embodiments, after the care giver is identified, the assessment tool may automatically render a graphical user interface, such as the assessment UI, that may present a prompt requesting input regarding an assessment data point acquired as part of acquisition of the assessment time series data.

At block 204, the process 200a includes acquiring, from a data storage device, a wearer profile record stored in the data storage device, the wearer profile record being created for the wearer, and the wearer profile record indicating a profile group from the plurality of profile groups to which the wearer is assigned and metadata related to the wearer. The wearer profile record is acquired for each wearer from the plurality of wearers in the care system.

The wearer may be assigned to the profile group from the plurality of profile groups based at least on a voiding pattern of the wearer, mobility of the wearer, and a cognitive ability of the wearer.

In some embodiments, a profile group is defined as shown in Table 1 below, wherein incontinence products are referred to as containment products.

TABLE 1
Definition of wearers in profile groups G1-G6.

| | |
|---|---|
| G1 | Those with incontinence who do not need care from others, although they may benefit |
| | from support in choosing appropriate containment products. |
| G2 | Those with either moderately reduced mobility or occasional mild confusion, and able to |
| | dress themselves, requiring occasional support but mostly achieving full toileting. |
| | Containment products provide reassurance in case toileting is not successful. |
| G3 | Persons with a combination of possible occasional confusion and either significantly |
| | reduced mobility or the inability to dress themselves, requiring assistance on many or |
| | most toileting occasions. A greater dependence on carer assistance, which may not |
| | always be available when needed, means the individual relies more often on containment |
| | than those in Group 2. |
| G4 | Those with a significant degree of confusion meaning a greater need for carer |
| | involvement to identify when to toilet as well as to support with the toileting itself, with |
| | more reliance on containment than those in Group 3. Mobility may be reduced to the |
| | degree that a maximum of one carer is required. The individual may or may not need |
| | assistance with dressing. Successful toileting is still achievable with a high degree of |
| | personalised care. |
| G5 | Persons for whom restricted mobility is the driver of T&CC care requirements. The need |
| | for an active mechanical lift in addition to support from a single carer to transfer to the |
| | toilet requires more carer time and results in a greater reliance on containment than those |
| | in Group 4. In addition, the individual may have any degree of confusion. Reduced |
| | mobility makes the individual likely to require support with dressing. Successful toileting is |
| | still achievable with carer support and a high degree of personalised care. |
| G6 | This population is defined by further reductions in mobility beyond Group 5 drives T&CC |
| | care needs. Toileting may still be possible with use of a passive mechanical lift and two |
| | carers to allow use of a bedside commode, but advanced immobility means that the |
| | majority of urine voids require containment. The individual is likely to need help with |
| | dressing. This degree of immobility may be accompanied by any degree of confusion. |

Group 1 encompasses care-independent persons, while Group 2 to Group 6 represent care-dependent population requiring different levels of toileting and containment care (T&CC) assistance.

At block 206, the process 200a includes sending the wearer profile record to an evaluation server. The processing at block 206 includes sending the wearer profile records of all wearers from the plurality of wearers in the care system to the evaluation server.

At block 208, the process 200a includes, in response to receiving the assessment time series for each wearer from the plurality of wearers, sending the assessment time series data to the evaluation server. The assessment time series data may be sent to the evaluation server via a secure network connection.

At block 210, the process 200a includes receiving, from the evaluation server, values of a plurality of evaluation parameters related to computer-generated evaluation of the process of use of the second set of one or more incontinence products worn by the plurality of wearers, based on processing by the evaluation server of the assessment time series data and the wearer profile record for each wearer from the plurality of wearers.

Further, as shown in FIG. 2B, the process 200a includes, at block 212, generating a computer representation of one or more values of the plurality of evaluation parameters corresponding to selected one or more profile groups for the care system from the one or more care systems. In some examples, the computer representation may be generated on a filter-based user interface including user-selectable filter fields for selecting the one or more profile groups from the plurality of profile groups, wherein the filter-based user interface is configured to present, in response to receiving user input via the user-selectable filter fields, the computer representation of the one or more values of the plurality of evaluation parameters corresponding to the selected one or more profile groups.

In some examples, the filter-based user interface may include user-selectable filter fields for selecting one or more care systems from the one or more care systems, and wherein the filter-based user interface is further configured to present, in response to receiving user input via the user-selectable filter fields, the computer representation of the one or more values of the plurality of evaluation parameters corresponding to the selected one or more profile groups and to the selected one or more care systems.

The filter-based user interface or the evaluation results UI, such as, e.g., the filter-based UI 122 of FIG. 1, may be configured to receive input via user-selectable filter fields so that a comparison of the effect of a product change on different profile groups, care systems, and/or wards or other units of the care system is enabled in an intuitive and user-friendly manner. In some embodiments, the filter-based user interface allows comparison between values of the plurality of evaluation parameters generated as part of assessment of respective different incontinence products, as discussed in more detail below.

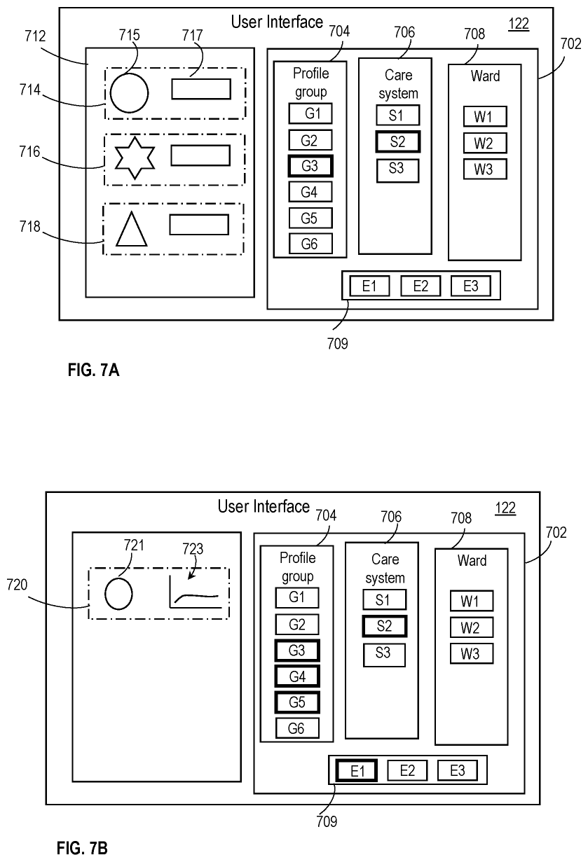

If indication of selection of more than one evaluation parameter is selected, a computer representation, generated responsive to the indication of the selection, may include a more complex graphical representation including representations of values of more than one, e.g. two, three, or more than three, evaluation parameters, which may be values computed for more than one profile group. Also, more than one care system, as well as one or more units, such as e.g. wards within the care system(s) may be selected. For example, when more than one care system is selected, a ward filter field, such as ward filter field 708 in FIGS. 7A and 7B, may include ward filter fields for each of the selected care systems. For example, a certain number of ward filter fields may be displayed for one of the selected care systems, and a different number of ward filter fields may be displayed for another of the selected care systems. A number of ward filter fields may depend on a number of wards in a respective care system.

The filter-based user interface may be rendered by the assessment tool on the computer device or on a different computer device. For example, the filter-based UI 122 of FIG. 1 may be rendered by the processor or processing circuitry 110 of the computer device 102. The filter-based UI may be rendered by the assessment tool on the computer device or on a different computer device. The filter-based UI 122 may be rendered by the assessment tool 112 and, in some implementations, the filter-based UI 122 may be part of the assessment UI 114 or it may be rendered by the assessment tool 112 separately from the assessment UI 114. Also, the assessment UI 114 may include a feature that can receive user input instructing the assessment tool 112 switch from the assessment UI 114 to the filter-based UI 122.

Alternatively, in some implementations, the filter-based UI 122 may be rendered on a computer device that is different from the computer device which renders the assessment UI 114.

In some embodiments, the computer representation indicates a comparison of the values of the plurality of parameters with respective values of the plurality of parameters associated with a different process of use of incontinence products of the second set. In this way, the computer representation may indicate, in a graphical format easily perceived by the user interacting with the filter-based UI 122, difference, in the form of the values of the plurality of parameters, between different processes of use of incontinence products of the second set. In such embodiments, values of the plurality of parameters for the different process of use of the incontinence products of the second set may have been previously generated by the evaluation server.

In some embodiments, the computer representation indicates a comparison of the values of the plurality of evaluation parameters—associated with the second process of use of the incontinence products of the second set—with respective values of the plurality of evaluation parameters associated with a first process of use of incontinence products of a first set. Methods in accordance with aspects of the present disclosure, such as the method(s) shown in FIGS. 2A and 2B, may be performed for assessment of an effect of a change from any set of incontinence products to any other set of incontinence products, such that a change from and to different sets may be assessed for different wearers in the care system. Thus, values of the plurality of evaluation parameters may be determined for an incontinence product that belongs to any set, and the evaluation server may compare the values to values of the plurality of evaluation parameters associated with a process of use of incontinence products of any other set. Thus, in some embodiments, methods in accordance with aspects of the present disclosure are used to generate respective values of the plurality of evaluation parameters in connection with incontinence products of the first set and incontinence products of the second set, wherein the incontinence products of the second set are being assessed for possible adoption by the care system, e.g., to be worn by one or more wearers instead of incontinence products of the first set.

FIG. 7A illustrates an example of a filter-based UI also referred to herein as an evaluation results UI, such as, e.g., filter-based UI 122 of FIG. 1 which may be rendered when the plurality of evaluation parameters, related to computer-generated evaluation of a second process of use of a second set of one or more incontinence products, are received from the evaluation server. The filter-based UI may be customizable. As shown in FIG. 7A, a selection portion or panel 702 on the right includes user-selectable filter fields including a profile group filter field 704 for selecting a profile group from the plurality of profile groups, a care system filter field 706 or selecting a care system, e.g., a nursing home, from the one or more care systems. The selection panel 702 may also include one or more user-selectable filter fields for receiving input indicating selection of a unit smaller than the care system in terms of a number of residents, such as e.g., a ward. For example, as shown in FIG. 7A, the selection panel 702 may include a ward filter field 708 configured to receive user input indicating selection of a ward within a selected care system. As an example only, the profile group filter field 704 is shown to include user-selectable filter fields for six groups G1, G2, G3, G4, G5, and G6, each corresponding to a respective profile group; the care system filter field 706 is shown to include user-selectable filter fields S1, S2, and S3, each corresponding to a respective care system; and ward filter field 708 is shown to include user-selectable filter fields W1, W2, and W3, each corresponding to a respective ward within a selected care system.

The user-selectable filter fields in the selection panel 702 may be rendered as a pull-down or other menu, and user input may be received indicating selection of any one or more out of one or more profile groups, one or more care systems, one or more wards, or one or more evaluation parameters. The selection panel 702 allows selecting, adding, or removing different user-selectable filter fields such that computer representation of different entities may be generated dynamically, in response to receiving input via the user-selectable filter fields.

In some examples, the user-selectable filter fields are configured to receive user input indicating selection of individual wearers in a care system, in which case the user may be enabled to visually perceive an effect on a wearer of a change of an incontinence product.

The user-selectable filter fields in the selection panel 702 may be configured to receive corresponding user input, which may be in the form of selection, e.g., via a touch, a click, or a voice command, of one or more user-selectable filter fields. A representation portion or panel 712 in FIG. 7A is configured, in response to the selection panel 702 receiving user input via the user-selectable filter fields, to present a computer representation of one or more values of the plurality of evaluation parameters corresponding to the selected one or more profile groups and/or one or more care systems. A ward, or another unit may also be selected via user-selectable filter fields provided on the selection panel 702.

In the example of FIG. 7A, the third profile group G3 is shown to be selected, and a second care system S2 is shown to be selected, as indicated via a bold line. The second care system S2 includes three wards W1-W3, and no wards are selected in the illustrated example, i.e. results may be presented for the entire second care system S2. It should be noted that the care systems S1, S2, and S3 may be associated with the same entity that manages and operates these care systems. In certain examples, a single care system may be available for selection, or there may be no need to select a care system since the evaluation results UI 122 may be presented via the assessment tool 112 in use by a single care system.

In some implementations, the selection panel 702 includes an evaluation parameter filter field 709 which is configured to receive an indication of a selection of one or more of the evaluation parameters, shown as three evaluation parameters E1, E2, and E3 as an example only. The evaluation parameter filter field 709 may be configured to receive an indication of a selection of fewer than three or more than three evaluation parameters. In certain examples, representations of all if the evaluation parameters used may be displayed for selection. If no specific evaluation parameter(s) are selected, the computer representation including values of all evaluation parameters may be generated. Alternatively, in some embodiments, the evaluation server may select evaluation parameters for being included in the computer representation by default. Any suitable number of evaluation parameters may be used.

Responsive to the indication of the selection of the one or more user-selectable filter fields, the representation panel 712 generates visual representations of values of evaluation parameters determined or computed for the third profile group G3 and the second care system S2. In this way, results of assessment of incontinence products of one or more sets (e.g., first and second sets, as in the illustrated embodiments) are shown in the form of the values of the evaluation parameters computed for persons such as wearers assigned to the third profile group G3 and residing in, or otherwise associated with, the second care system S2.

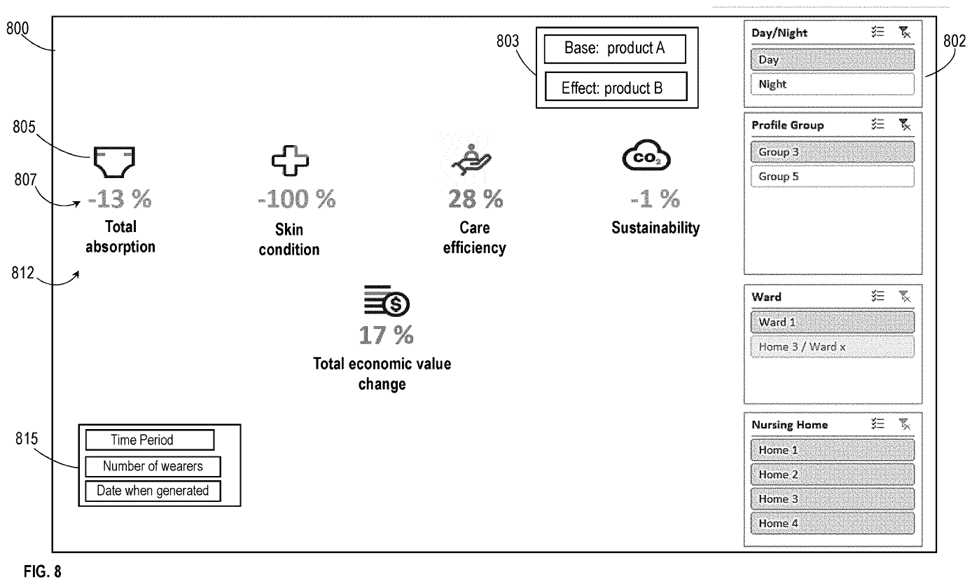

The representation panel 712 in FIG. 7A illustrates a first computer representation 714 of one or more values of a first evaluation parameter from the plurality of evaluation parameters, a second computer representation 716 of one or more values of a second evaluation parameter from the plurality of evaluation parameters, and a third computer representation 718 of one or more values of a third evaluation parameter from the plurality of evaluation parameters. The computer representation of one or more values of the plurality of evaluation parameters may includes a first icon representing a corresponding evaluation parameter from the plurality of evaluation parameters and a second icon related to the first icon and representing a value of the corresponding evaluation parameter. For example, using the first representation 714 in the representation panel 712 as an example, the first representation 714 includes a first icon 715 representing a corresponding evaluation parameter and a second icon 717 related to the first icon 715 and representing a value of the corresponding evaluation parameter. The second icon 717 may include a value in a numerical, graphical, or other format, or a combination of different formats. FIG. 8, discussed below, illustrates examples of icons.

The second computer representation 716 and the third computer representation 718 similarly each include related icons, first representing an evaluation parameter and a second, related to the first, icon representing a value of the evaluation parameter represented by the first icon.

FIG. 7B illustrates an example of the filter-based UI 122 that received an indication of a selection of an evaluation parameter E1 via the evaluation parameter filter field 709. In this example, the filter-based UI 122 also shown to have received an indication of a selection of three profile groups G3, G4, and G5 via the profile group filter field 704. Responsive to the indication of the selection, a computer representation 720 may be generated that includes a first icon 721 representing the selected evaluation parameter E1 and a second icon or, in this example, a graphical view, 723 related to the first icon 721 and representing values of the evaluation parameter E1. The values of the evaluation parameter E1 may be shown for three profile groups G3-G5, respectively, and the values may be represented, e.g., as a graph illustrated schematically in FIG. 7B.

It should be noted that features of FIGS. 7A and 7B are shown for illustration purposes only. A filter-based UI, generated and automatically rendered using techniques in accordance with the present disclosure, may include any features that may be positioned in different portions of the filter-based UI in various ways.

In some embodiments, the filter-based UI includes interactive features. For example, one or more of the graphical elements generated on the filter-based UI 122 shown in FIGS. 7A and 7B may be interactive. For example, the first icon 721 representing the selected evaluation parameter E1 may be interactive such that it may be selectable and, responsive to the selection, additional information on the selected evaluation parameter E1 may be displayed. The filter-based UI 122 may generate different views.

FIG. 8 illustrates a snapshot of an example of a filter-based UI 800 in accordance with aspects of the present disclosure. In FIG. 8, a selection portion or panel 802 on the right includes user-selectable filter fields including filter fields for receiving input regarding selection of a day type of an incontinence product of a certain set, a night type of the incontinence product of a certain set, a profile group, a ward, and a care system such as a nursing home. The filter-based UI 800 also includes a representation portion or panel 812, in the portion of the filter-based UI 800 separate from the selection panel 802. The representation panel 812 displays, in response the selection panel 802 receiving user input via the user-selectable filter fields, presenting a computer representation of values of a plurality of evaluation parameters for the selected day type of an incontinence product, Group 3, ward 1, and care systems such as nursing homes 1-4. In this example, the representation panel 812 displays computer representations for evaluation parameters including a total absorption change (e.g., per year, month or another time period), skin health (defined, e.g., as a change per year, month or another time period in a number of wearers in need of skin care), care efficiency (defined as, e.g., as a change per year, month or another time period in staff of care giver's time), sustainability (defined, e.g., as a change per year, month or another time period in CO₂ usage by the care system), and a total economic value change. For example, an icon 805 is a computer representation of the total absorption change, and an icon 807 is computer representation of a value of the total absorption change. The icon 805, and any other icon or another graphical feature, may be an interactive object such that, in response to a user input indicating its selection, the filter-based UI 800 is triggered to switch to another user interface view that may overlay the current user interface view and display UI features that allow viewing evaluation parameter values related to the selected evaluation parameter, such as, in this example, the total absorption change.

The filter-based UI 800 also displays, in a product indication panel 803, a baseline or base incontinence product and an incontinence product compared to the base product, effect of a change to which is evaluated. For illustration purposes only, the base product is denoted as product A or a set of products A, and the “effect” product is denoted as product B or a set of products B. In embodiments described herein, the base product is referred to as an incontinence product of a first set and the product effect of a change to which is evaluated is referred to as an incontinence product of a second set.

The product indication panel 803 may be a static digital object that can be displayed, e.g., when assessment time series data has been acquired for two incontinence products of different types—e.g., based on a process of use of a first set of one or more incontinence products and of a second set of one or more incontinence products. For example, when the assessment time series data is acquired, a current stage may be indicated, e.g, via stage field in the assessment UI 114 of FIG. 1. Thus, the computer device through which the assessment time series data is acquired, and the evaluation server receiving this data, will treat the assessment time series data accordingly and the filter-based UI may display a computer representation indicating, in connection with the representation of the values of the evaluation parameters, which product set was used as a base product, e.g., a first set of one or more incontinence products, and which product set was used as a comparison or effect product, e.g., a second set of one or more incontinence products.

In some cases, one type of a base product, e.g., an incontinence product of a first set, may be used as a base for comparison with more than one assessed effect products, e.g., an incontinence product of a second set. The “second” set, as in other examples herein, may mean a set including incontinence products having at least one difference from incontinence products of the first set, or having a difference in a process of use among the first and second sets. The product indication panel 803 may be an interactive digital object through which user input may be received indicating a selection of a base incontinence product set and another, effect incontinence product set an effect of a change to which is to be displayed on the filter-based UI 800. Although not shown in FIG. 8, the product indication panel 803 may be configured to receive user input regarding selection of more than one pair of base-effect product sets, such that the representation panel 812 can display a computer representation of values of a plurality of evaluation parameters for the selected base-effect product sets. For example, when respective processes of use of different first and second product sets were assessed for different wearers in a care system, the product indication panel 803 may allow a user to submit input indicating selection of several base and effect products. As an example implementation, the product indication panel 803 may be in the form of a pull-down menu from which the user can select base-effect product sets pairs corresponding to the first and second products sets. As another possible implementation, the product indication panel 803 may, in its default configuration, not require a selection of base and effect products, such that the representation panel 812 may by default display computer representations of values of a plurality of evaluation parameters for all assessed, e.g., for a care system or a group of related care systems, base and effect product sets. A user may then submit input via the product indication panel 803, to instruct the representation panel 812 to display results regarding a certain pair of base and effect product sets from all of the assessed base and effect product sets or regarding more than one pair of base and effect product sets, depending on what assessment has been done for a care system or a group of related care systems.

In the example of FIG. 8, the care efficiency and the total economic value change are greater than when a respective base incontinence product set was used, thus demonstrating a superiority of the incontinence product set being assessed such as of incontinence products of a second set. In response to such determination, a suitable action, indication, and/or a recommendation may be initiated or generated, in accordance with embodiments of the present disclosure. In some embodiments, values of evaluation parameters may be compared to corresponding thresholds, to determine whether a change in the value, reflecting a temporary, during an assessment time, switch to another incontinence product, is indicative of a sufficient improvement in the value that warrants a more permanent change to the incontinence product set being evaluated.

In some embodiments, ranges of one or more evaluation parameters may be determined to define care requirement groups. Optionally, the method may involve computer generated representation of one or more care requirement groups. Thus, the care requirement groups may be used for a simplified representation of one or more evaluation parameters. For example, in response to receiving first and second assessment time series data, the generated values of the plurality of evaluation parameters may indicate a move from a first care requirement group to a second care requirement group. For example, the evaluation parameter care efficiency, defined as the required staff of care giver's time, could be used to define care efficiency groups, where each care efficiency group corresponds to a range of the evaluation parameter care efficiency. In response to receiving the first and second assessment time series data, the generated values of the plurality of evaluation parameters may indicate a move from a first care efficiency group to a second care efficiency group. This represents on a group level a change in the required staff or care giver's time, and thus provides a simplified view of the evaluation parameter care efficiency. Similarly, absorption groups may be defined by applying ranges to the evaluation parameter absorption.

In addition, in some examples, as shown in FIG. 8, the representation panel 812 may include an information panel 815 indicating a time period for which the assessment results in the representation panel 812 is displayed, a number of wearers such as residents regarding which the assessment results are generated, and a date when the assessment results were generated.

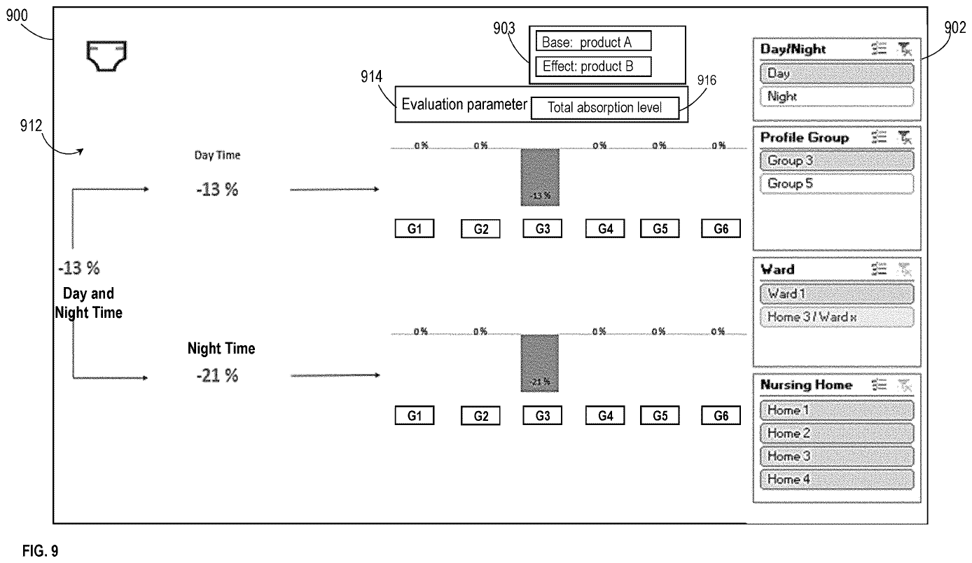

FIG. 9 illustrates a snapshot of an example of a filter-based UI 900 in accordance with aspects of the present disclosure, wherein the filter-based UI 900 includes computer representations of values, for a selected profile group 3 and a day and night types of incontinent products of a certain incontinent product set, of the evaluation parameter such as the total absorption level. The filter-based UI 900 may be rendered, for example, when the total absorption level evaluation parameter is selected though an evaluation parameter selection field 914 which may include an input field 916. The filter-based UI 900 may in some cases be rendered in response to a user input indicating, in this example, a selection of the total absorption level evaluation parameter, e.g., through a user interface such as e.g. filter-based UI 800 of FIG. 8. Thus, for example, the computer representation 805 corresponding to the total absorption level evaluation parameter may be interactive such that it may be activated to trigger a display of a representation portion or panel 912, or a panel that would overlay the representation panel 812 of FIG. 8. The evaluation parameter selection field 914, which may be displayed regardless of the manner in which the filter-based UI 900 is rendered, may be used to receive user input indicating selection of one or more other evaluation parameters, e.g., via the input field 916.

In FIG. 9, a selection portion or panel 902 includes user-selectable filter fields and the representation portion or panel 912 displays values of the total absorption level evaluation parameter, separately for a day type of an incontinence product of a second set, a night type of the incontinence product of the second set, and a combined value of the values for the day and night types of the incontinence product of the second set. In some cases, a value of the total absorption may be calculated using a Rothwell scale, or Rothwell system that is applied to incontinence care products, and is a scale that shows how absorbent a particular incontinence product is, and how much liquid it can absorb and hold before it is likely to leak due to overfill.

In the illustrated example, the representation panel 912 also illustrates respective graphs for the day and night types of the incontinence product of the second set. In the graphs, all six groups G1-G6 are indicated and the values of the total absorption level evaluation parameter for the selected group 3, G3, are shown. The graphs are shown by way of example only, as any other computer representation may be used to visualize values of the evaluation parameters.

The filter-based UI 900 may include a product indication panel 903, which may be similar to product indication panel 803 of FIG. 8. The product indication panel may be interactive so as to allow selecting a base or baseline product or product set, and a product or set an effect of a change to which, as compared to the baseline product, is desired to be visualized.

In some embodiments, the method in accordance with aspects of the present disclosure further includes generating a second computer representation of a configuration of the care system and displaying on the second computer representation a location of the wearer from the plurality of wearer in the care system. The second computer representation may include the values of the plurality of evaluation parameters related to computer-generated evaluation of the second process of use of the incontinence products of the second set worn by the wearer. The second computer representation may additionally or alternatively include the values of the plurality of evaluation parameters related to computer-generated evaluation of the first process of use of the incontinence product of the first set worn by the wearer. The second computer representation may be generated on the computer device such as, e.g., computer device 102 of FIG. 1 or on a display associated with the computer device 102. The second computer representation may be generated on the filter-based UI or on another UI rendered by the processing circuitry of the computer device.

In some embodiments, an incontinence product of the second set may include a second wireless sensor configured to measure voiding activity of the wearer using the incontinence product of the second set as measured second voiding data, and configured to send the measured second voiding data to a digital diary in communication with the data storage device, so that the wearer profile record is updated to store the measured second voiding data. The digital diary may be part of an assessment tool, e.g., assessment tool 112 of FIG. 1. Thus, the assessment tool may receive the measured second voiding data and it may send the measured second voiding data to the data storage device 106 that records the measured second voiding data in a respective profile record stored for the wearer. For example, with reference to system 100 of FIG. 1, the measured second voiding data may be stored as part of the metadata, e.g., metadata 132 of a wearer profile record in wearer profile records storage 124. Furthermore, the measured second voiding data may be used for computer-based assignment of the wearer of the incontinence product of the second set to a profile group from the plurality of profile groups. The assessment tool may receive the assessment time series data and the measured second voiding data, and it may integrate the assessment time series data with the measured second voiding data, and send the assessment time series data and the measured first voiding data to the evaluation server.

Similarly, an incontinence product of the first set may include a first wireless sensor configured to measure voiding activity of the wearer using the incontinence product of the first set as measured first voiding data, and configured to send the measured first voiding data to a digital diary in communication with the data storage device, so that the wearer profile record is updated to store the measured first voiding data. As discussed above, the digital diary may be part of assessment tool 112 of FIG. 1. The measured first voiding data may be handled similar to the measured second voiding data discussed above.

Referring back to FIG. 2B, at block 214, the process 200a further includes receiving, from the evaluation server, a recommendation for the care system regarding the process of use of the incontinence products of the second set. The recommendation may include a recommendation for a modification to at least one step from the plurality of second steps of the second process of use of the one or more incontinence products of the second set. As another example, the recommendation may include a recommendation for a modification to a configuration of an incontinence product of the second set.

At block 216, the process 200a further includes receiving, from the evaluation server, an indication to the care system, the indication recommending that the care system uses incontinence products of the second set. The indication may be received in any suitable format and via any suitable computing device. As used herein, the terms “indication” and “recommendation” are used interchangeably. In some embodiments, the process 200a may further include, in response to receiving the indication, automatically sending an order to acquire a plurality of incontinence products of the second set, as shown at block 217 of FIG. 2B. Thus, when the method in accordance with aspects of the present disclosure determines, based on processing by the evaluation server of the assessment time series data and the wearer profile record, and based on computing values of the plurality of evaluation parameters, that it is more advantageous for the care system to use incontinence products of the second set, an automatic order for the plurality of incontinence products of the second set may be sent to a manufacturer or distributor. The incontinence products of the second set may thus be shown to be superior to incontinence products of the first set.

In some embodiments, the assessment tool 112 is configured to ensure that an indication of an action is received in connection with the received values of the plurality of evaluation parameters. In other words, once the values of the plurality of evaluation parameters are received from the evaluation server, the assessment tool 112 monitors whether an input is received from a user in the care system regarding the values of the plurality of evaluation parameters, and thus regarding the evaluation of incontinence products of the second set. For example, as shown in FIG. 2B, a notification may be generated after a certain period of time, at block 218. The notification may be generated by the computer device 102 or it may be received from the evaluation server 104 or generated based on an instruction received from the evaluation server 104. The notification requests an indication of an action regarding the computer-generated evaluation of the process of use of the incontinence products of the second set worn by the wearer. For example, if, after the certain period of time, the filter-based UI or another UI rendered by the assessment tool 112 did not receive an indication of an action regarding the evaluation of the process of use of the incontinence product of the second set, the user of the assessment tool 112 may be automatically instructed to submit an indication of an action. The indication of an action may be, e.g., an indication to order incontinence products of the second set, an indication of a decision not to proceed with use of incontinence products of the second set, an indication of a further inquiry regarding the incontinence products of the second set, or any other indication that indicates a certain decision regarding the plurality of evaluation parameters.

In example embodiments, the processing at blocks 214, 216, 217, and 218 of FIG. 2B may be performed as part of the processing at block 211 of FIG. 2A.

In some embodiments, an incontinence product of the second set may be selected based on sensor data acquired from a test incontinence product, the test incontinence product being used by the wearer during an information collecting time period. The test incontinence product may be a type of an incontinence product that is configured to be worn by a wearer during a certain period of time, for purposes of assessing wearer's voiding behaviour and other features. The test incontinence product performs functions of a regular, i.e. not used for testing, incontinence product.

Methods according to the embodiments described herein for the computer device 102, such as e.g, methods 200, 200a discussed above may be respectively implemented using e.g. a computer program product, or a computer program, including instructions, i.e., software code portions, which, when executed on at least one processor, cause the at least one processor to carry out the actions described herein, as performed by the computer device 102. The computer program product may be stored on a computer-readable storage medium, e.g. a disc, a universal serial bus (USB) stick or similar. The computer-readable storage medium, having stored thereon the computer program product, may include the instructions which, when executed on at least one processor, cause the at least one processor to carry out the actions described herein, as performed by the computer device 102.

FIG. 3 illustrates an example of communications between components of a computer system such as the system 100 of FIG. 1. As shown in FIG. 3, the computer device 102 may receive or acquire assessment time series data, e.g., via a graphical UI of an assessment tool. The computer device 102 receives a wearer profile record, associated with a profile identifier, from the data storage device 106. The computer device 102 then sends the wearer profile record and the assessment time series data to the evaluation server 104, which may be performed separately or simultaneously. The computer device 102 then receives values of a plurality of evaluation parameters from the evaluation server 104. The evaluation server 104 may instruct (shown as an arrow 302) the computer device 102 to render a filter-based UI, such as the filter-based UI 122 of FIG. 1, that is configured for interaction with a user regarding analysis of values of a plurality of evaluation parameters displayed as computer-representations on the filter-based UI.

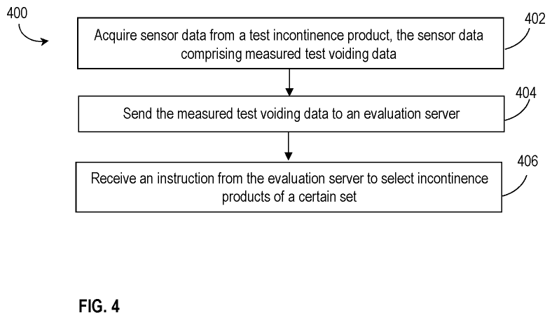

FIG. 4 illustrates a method or process 400 of selecting one or more incontinence products of the second set in accordance with some embodiments of the present disclosure. The method may be implemented by a computer device, e.g., computer device 102 of FIG. 1 or another computer device in communication with an evaluation server such as, e.g., evaluation server 104 of FIG. 1.

At block 402 of FIG. 4, sensor data may be acquired from a test incontinence product. The test incontinence product may include a wireless sensor configured to measure voiding activity of the wearer using the test incontinence product as the sensor data including measured test voiding data, and configured to send the measured test voiding data to the evaluation server. The test incontinence product may be a disposable incontinence product, e.g., in the form of disposable underwear. The wireless sensor associated with the test incontinence product may be a thin, discrete component for collecting voiding information and configured to be placed in the test incontinence product in a manner that does not cause a discomfort to the wearer. For example, the test incontinence product may include a compartment configured to removably house the wireless sensor. The wireless sensor may be positioned within the test incontinence product so as to measure amount, frequency, and other features related to liquid and solid discharge.

The sensor data such as the measured test voiding data may be acquired during an information collecting time period of a suitable duration. In some embodiments, the information collecting time period is 72 hours. In some embodiments, the information collecting time period is 24 hours, or 48 hours, or longer than 72 hours.

At block 404, the measured test voiding data is sent to an evaluation server. The wireless sensor may be configured to record and transmit the test voiding data to a device or server, such as an evaluation server.

In some cases, the test incontinence product may be associated with or include a logger system or kit including a reusable logger and charger. The logger may be communicatively coupled to the wireless sensor and configured to record the test voiding data acquired by the wireless sensor and transmit the test voiding data to an evaluation server. The charger is configured to charge the logger when the logger is not in use. The logger may be a reusable computing device configured and sized to be removably positioned in the test incontinence product, e.g., in a compartment configured to house the wireless sensor and the logger. The logger may alternatively be removably coupled to an outer surface of the test incontinence product, e.g., to a front surface, in the manner that does not interfere with the wearer's use of the test incontinence product. The wireless sensor may be associated with an identifier or an ID uniquely identifying the wireless sensor. In implementations in which the logger is used, the logger may be associated with a unique ID identifying the logger such that a server that receives data transmitted by the logger can determine that the sender of the data.

In some embodiments, the test incontinence product includes a TENA Smart Care Identifi™ product (Essity) that employs smart sensor technology. It should be appreciated however that embodiments in accordance with the present disclosure are not limited to any specific test incontinence product, and any type of an incontinence product, included or associated with a sensor such as a wireless sensor, may be used for test purposes, i.e. to acquire, using the sensor, voiding data collected as a person is wearing the test incontinence product during a certain time period.

The evaluation server may integrate the measured test voiding data with other information related to the wearer of the measured test voiding data and information acquired from a care system in which the wearer may be residing. The information on the wearer may be acquired by the evaluation server from memory such as, for example, data storage device 106 of FIG. 1. For example, in some embodiments, the information on the wearer may be stored in a respective profile record of the records stored in a profile records storage.

The evaluation server may analyze the measured test voiding data, along with the other information, and determine which type of an incontinence product is appropriate for the wearer. The evaluation server may also determine whether any adjustments to a process of use of an incontinence product suitable for a wearer.

Thus, at block 406 of FIG. 4, an instruction may be received from the evaluation server to select an incontinence product of a certain set. In some embodiments herein, the instruction may indicate a selection of the incontinence product of the second set as the most appropriate incontinence product for the wearer. The instruction may be received via a graphical user interface or in any other manner. Regardless of its specific format, the instruction may be received in the manner that prompts an action from a care facility.

In some examples of the present disclosure, assessment time series data is acquired in connection with a first process of use of a first set of one or more incontinence products and a second process of use of a second set of one or more incontinence products. Values of a plurality of evaluation parameters may be generated indicating a comparison of the first process of use of the one or more incontinence products of the first set worn by a wearer and the second process of use of the one or more incontinence products of the second set worn by the wearer. This allows transforming into a computer-generated numerical and/or other form and visualizing difference between incontinence products of different types.

In some examples, as described, e.g. in connection with FIGS. 2A and 2B, a method in accordance with aspects of the present disclosure may be performed at least in part remotely i.e. when at least some of its steps or actions are performed by a remote computer system e.g. an evaluation server. For example, the assessment tool 112 of system 100 as described herein, in operation, may be executed on the computer device 102 and may also rely on server processing for processing of assessment time series data and determination of values of evaluation parameters. Thus, for example, evaluation server 104 may perform the processing, along with other data, of the assessment time series data and determination of values of the evaluation parameters in accordance with aspects of the present disclosure.

In some examples, methods in accordance with aspects of the present disclosure may be performed locally, e.g., on one or more computer devices or systems associated with and controlled by a care system. For example, at least one hardware processor e.g. processing circuitry of a computer device such as, e.g., computer device 102 of FIG. 1, may perform a method in accordance with aspects of the present disclosure. For example, the assessment tool 112 may be executed on the computer device 102, using the processing circuitry of the computer device 102. The computer device 102 may communicate with a server and/or other devices or systems during the performance of the method, such that data may be acquired by the assessment tool 112, for example, from a remote storage device and/or from the evaluation server 104.

Furthermore, in some examples, methods in accordance with aspects of the present disclosure may be performed remotely, e.g., from a remote computer system such as e.g. evaluation server 104 of system 100. In such examples, for example, the assessment tool 112, accessible via the computer device 102, may be executed at least in part or wholly on the evaluation server 102.

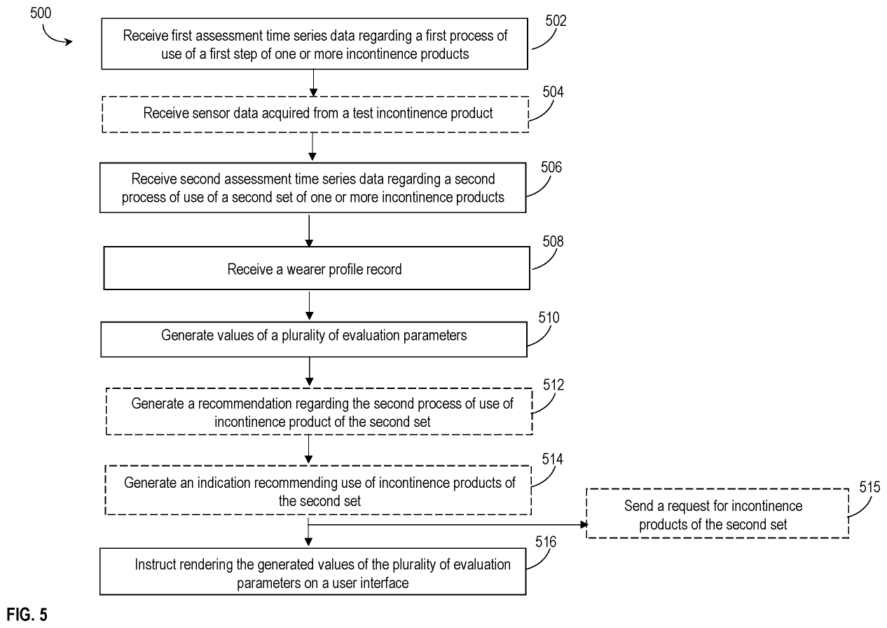

FIG. 5 illustrates an example of a computer-implemented method or process 500 that includes executing at least one processor of a computer system to perform steps or acts of the process 500. The least one processor of the computer system may be operated to execute computer-executable instructions stored in memory of the computer system. The computer system may be an evaluation server such as, e.g., evaluation server 104 of FIG. 1 such that at least one processor 144 of the evaluation server 104 may be operated to execute computer-executable instructions stored in memory 146 of the evaluation server 104 and/or in another storage device. The process 500 is described herein as being performed by the evaluation server 104. In some examples, however, the computer system may be computer device 102 of FIG. 1.

The process 500 may include processing acts that are similar to some of the acts of processes 200 (FIG. 2A) and 200a (FIG. 2B), and description of certain features is not repeated herein for the sake of brevity. The steps or acts of the process 500 can be performed in any suitable order which can be different from the illustrated order. Optional actions are shown using dashed lines.

At block 502, the process 500 includes receiving, for each wearer from a plurality of wearers in a care system, during a first assessment time period, first assessment time series data regarding a first process of use of one or more incontinence products of a first set worn by the wearer.

The first assessment time series data may be acquired during the assessment time period. The data may be acquired at time points that are equally spaced in time, e.g., every 2 hours or every 3 hours or every 4 hours. The time points may also be unequally distributed over the duration of the assessment time period. For example, at nighttime, fewer data points may be acquired at more sparsely spaced time points.

Non-limiting examples of the first assessment time series data include one or more out of skin assessment, a change of an incontinence product of the first set, feces in the incontinence product of the first set, a toilet visit, a cleansing and protection event, an occurrence of urine leakage, a clothes change event, a linen change event, an application of a day type of the incontinence product of the first set, or an application of a night type of the incontinence product of the first set.

At block 504, optionally, after the first assessment time series data is received, sensor data acquired from a test incontinence product may be received. As discussed above, the test incontinence product may be used by the wearer during an information collecting time period, and the test incontinence product includes a wireless sensor configured to measure voiding activity of the wearer using the test incontinence product as the sensor data including measured test voiding data. The test incontinence product is configured to send the measured test voiding data to the evaluation server. For example, as discussed above, the test incontinence product may send the measured test voiding data using the wireless sensor and/or a logger. The measured test voiding data may be used to select one or more incontinence products of the second set, e.g., as a potential replacement of the incontinence products of the first set. In other words, the measured test voiding data may be used to determine whether it is worthwhile evaluating whether the second set of one or more incontinence products may be more suitable for a certain wearer than the first set of one or more incontinence products. For example, if incontinence products of the first set are not sufficiently absorbent for the wearer, it may be decided that another set of one or more incontinence products may be a better fit for the wearer. Thus, an assessment in accordance with examples of the present disclosure may follow, e.g., to compare respective processes of use of a second set of one or more incontinence products and of the first set of one or more incontinence products.

The processing at block 504 may be optional since the second set of one or more incontinence products may be selected in any suitable way, and examples herein are not limited to a specific way in which the second set of incontinence products is selected.

At block 506, the process 500 includes receiving, for each wearer from the plurality of wearers, during a second assessment time period, second assessment time series data regarding a second process of use of a second set of one or more incontinent products worn by the wearer. The wearer is the same person that was previously wearing the incontinent products of the first set, such that the assessment time series data is acquired, regarding processes of use of two sets of products, per wearer. The second assessment time series data may be acquired to computationally determine whether use of the incontinent products of the second set, as compared to the use of the incontinent products of the first set, would be more advantageous to the wearer of the product(s) and and/or the care system. A conventional method of comparing advantages and shortcomings of incontinent products of different types does not involve computer-based derivation of values of evaluation parameters as performed in accordance with aspects of the present disclosure, while the aspects thus advantageously allow making more informed decisions regarding a choice and use of incontinent products.

The second assessment time series data may be acquired for a certain duration of time such as the second assessment time period, which may be the same duration of time as the duration of time used to acquire the first assessment time series data. Thus, in some examples, the first assessment time period and the second assessment time period may have the same duration of time, or a similar duration of time. It should be noted that the first assessment time period and the second assessment time period may not have an exact same duration of time. For example, additional data may be acquired during one or both of the assessment time period, e.g., when a collection of data at a scheduled time point is missing or for any other reasons. Also, differences in the first and second processes of use may require acquisition of different amount of data. The second assessment time series data may be acquired at certain time intervals. Time points at which the second assessment time series data is acquired may be predefined. The time points may be the same as those used for acquisition of the first assessment time series data, though in certain cases there may be some difference, as discussed above. Also, a user such as a caregiver may be able to override the predefined or preset time points, e.g., when a product check is required at a time not coinciding with a predefined time point.

In certain examples, the first assessment time series data and the second assessment time series data may be received via an assessment user interface of an assessment tool, such as, e.g., the assessment tool 112 of FIG. 1.

Non-limiting examples of the second assessment time series data include one or more out of skin assessment, a change of an incontinence product of the second set, feces in the incontinence product of the second set, a toilet visit, a cleansing and protection event, an occurrence of urine leakage, a clothes change event, a linen change event, an application of a day type of the incontinence product of the second set, or an application of a night type of the incontinence product of the second set.

The assessment parameters related to the use of the incontinence products of the first set worn by the wearer and the assessment parameters related to the use of the incontinence products of the second set worn by the wearer may be the same.

In some examples, the assessment parameters related to the use of the incontinence products of the first set worn by the wearer and the assessment parameters related to the use of the incontinence products of the second set worn by the wearer may differ. For example, differences between the first and second sets may require acquisition of different types of assessment data, such that there may be a resulting difference between the assessment parameters employed in connection with the first set and the second set, respectively. Also, when there are some differences in the respective processes of use of the first and second sets of incontinence products, different type of assessment data may be acquired regarding a process of use of one of these sets relative to another one of the first and second set. However, even if there may be a difference in the assessment parameters, the first assessment time series data and the second assessment time series data is comparable.

At block 508, the process 500 includes receiving a wearer profile record being created for the wearer, the wearer profile record indicating a profile group from the plurality of profile groups to which the wearer is assigned, and metadata related to the wearer. The wearer profile record may be received, e.g., from a data storage device. The wearer profile record may be received directly from the data storage device, e.g., data storage device 106 of FIG. 1, or via a computer device such as e.g. computer device 102 which is configured to access the data storage device.

The first assessment time series data includes assessment values of assessment parameters related to the use of the incontinence products of the first set worn by the wearer, and the second assessment time series data includes assessment values of assessment parameters related to the use of the incontinence products of the second set worn by the wearer. The first process of use and the second process of use each include a plurality of steps. The first and second processes may have the same or similar steps, such that respective data acquired as a result of the first and second processes is comparable. The steps of both the first and second processes may include checks of a condition of the wearer and/or a state of an incontinence product worn by the wearer, e.g., partially undressing the wearer, taking of off the wearer or assisting the wearer to take of a used (soiled) incontinence product, cleansing the wearer's body part affected by the used incontinence product, applying a cream or another composition to the wearer's skin, preparing a new incontinence product to be administer to the wearer, and administering the new incontinence product to the wearer. Any steps or sub-steps may be included additionally or alternatively. The process of use of an incontinence product may also include changing linens, manual or automatic monitoring the state of the product such as checking whether the currently worn product remains dry or whether it has been soiled, etc. The check may not include a change of clothes or linens, i.e. when it is not required. A plurality of steps of the process of use may be defined at the evaluation server. Also, a care system may develop and employ a process of use for a certain set or more than one sets that is suitable for wearers, care workers, type(s) of the product used, particulars of the care system, etc. The process of use of an incontinence product of a certain set may be adjusted, as discussed herein.

As in other examples discussed herein, the wearer may be assigned to the profile group from the plurality of profile or care groups based at least on a voiding pattern of the wearer, mobility of the wearer, and a cognitive ability of the wearer. For example, the wearer may be assigned to one of the six profile groups shown in Table 1, above. However, any other number of profile groups, including less than six or greater than six may be employed in the methods in accordance with aspects of the present disclosure.

Furthermore, for each profile group from a plurality of profile groups, a step from the plurality of steps is associated with an estimated duration of time required to perform the step, e.g., as shown in profile group table 134 of FIG. 1. More specifically, persons assigned to different profile groups may require different amount and type of assistance regarding wearing incontinence products. Thus, depending on the profile group to which the wearer is assigned based on the wearer's ability to use an incontinence product and other factors, different amount of work, defined as an amount of time herein, is required to perform each step of the process of use of an incontinence product. For example, it may take less time to take off the used incontinence product and wash the wearer if the wearer has a moderately reduced mobility or occasional mild confusion, as compared to wearers having reduced mobility, e.g., as shown in Table 1 for wearers of Group 2 and Group 5 or 6.

As used herein, a process of use of an incontinence product is considered to be performed by a carer, caregiver or any other person other than the wearer. It should be understood however that, depending on the wearer's physical and mental abilities, the wearer may take part in the change and administration of a new incontinence product, thereby participating in the process of use of an incontinence product. Also, for wearers assigned to a profile group in which persons are capable of independent toileting, e.g., Group 1 in Table 1, an assessment and evaluation process in accordance with embodiments of the present disclosure may be performed as the wearer is performing the process of use of an incontinence product.

Regardless of the specific way in which a process of use of an incontinence product is performed in practice, it is defined to include a plurality of steps each being associated with an estimated duration of time required to perform that step, and the estimated duration of time may be different for different profile groups.

The block 510, the process 500 includes, in response to receiving the first and the second assessment time series data and receiving the wearer profile record for each wearer from the plurality of wearers, generating values of a plurality of evaluation parameters indicating a comparison of the first process of use of the first set of one or more incontinence products worn by the wearer and the second process of use of the second set of one or more incontinence products worn by the wearer, based on processing the first assessment time series data, the second assessment time series data, and the wearer profile record for each wearer from the plurality of wearers. The processing at block 510 may include also receiving information or data on one or more of product data, care system settings, or evaluation settings. For examples, data from product data storage 147 (FIG. 1) and care system settings 135 (FIG. 1), and evaluation settings 139 (FIG. 1) may be received. The values of the plurality of evaluation parameters may be estimated based on one or more of the product data, the care system settings, or the evaluation settings. The values of a plurality of evaluation parameters may be generated based on the first and second assessment time series data and based on data on one or more of the product data, the care system settings, or the evaluation settings.

The values of the plurality of evaluation parameters may indicate a comparison between a first plurality of evaluation parameters related to computer-generated evaluation of the first process of use of the first set of one or more incontinence products, and a second plurality of evaluation parameters related to computer-generated evaluation of the second process of use of the second set of one or more incontinence products. The values of the plurality of evaluation parameters may indicate a ratio between the first plurality of evaluation parameters and the second plurality of evaluation parameters.

In some embodiments, the comparison between the first plurality of evaluation parameters and the second plurality of evaluation parameters may indicate the effect on the values of the evaluation parameters of a difference between certain features of the incontinence product of the second set versus the incontinence product of the first set. For example, a presence of a leakage barrier, a type of a top sheet material, a chance in an absorption level of the product, a chance in a body fastening type (e.g., an insert pad, an open diaper, or a belted diaper), which may be different between the incontinence products of the second and first sets, may be evaluated in the form of the effect of such change(s) to the values of one or more evaluation parameters.

At block 512, a recommendation may be generated for the care system regarding the second process of use of the incontinence product of the second set. The recommendation may include a recommendation to use the incontinence product of the second set in the care system.

In certain examples, the recommendation may include recommendation for a modification to at least one step from the plurality of steps of the second process of use of an incontinence product of the second set. In certain examples, the recommendation may include a recommendation for a modification to a configuration of an incontinence product of the second set. The recommendation for the care system may be generated using the sensor data acquired from the test incontinence product. The sensor data, such as measured test voiding data, acquired from the test incontinence product, may be used in addition to the assessment time series data to determine whether to recommend use of the incontinence product of the second set to the care system.

In some cases, the recommendation may be generated solely based on the sensor data acquired from the test incontinence product. Such recommendation may be used to make a decision regarding assessment and evaluation of the incontinence product of the second set to the care system. For example, a preliminary recommendation to assess and evaluate the incontinence product of the second set may be generated based on the sensor data acquired from the test incontinence product. A final recommendation may subsequently be generated, after values of the evaluation parameters have been determined for assessment and evaluation of the incontinence product of the second set.

At block 514, an indication to the care system may be generated, the indication recommending that the care system uses incontinence products of the second set. The indication may be generated using the sensor data acquired from the test incontinence product.

In some embodiments, as shown at block 515, the process 500 optionally includes, when the value of at least one of the plurality of evaluation parameters has reached or exceeded a threshold, automatically transmitting, to an external computer, a request for an amount of incontinence products of the second set to be delivered to the care system. For example, when a value of an evaluation parameter, e.g., a total economic value change i.e. increase in cost savings by the care system, exceeds a certain threshold, an automatic decision may be made regarding use of incontinence products of the second set by the care system, which may thus replace incontinence products of the first set. The automatic decision may involve sending an automatic order for a certain amount of incontinence products of the second set. The automatic decision may prompt initiation of other actions, e.g., an indication of a required training for care givers, an indication of a change to a process of use of incontinence products of the second set, and other actions.

At block 516, the evaluation server may instruct rendering the generated values of the plurality of evaluation parameters on a user interface. For example, the evaluation server may instruct generating or rendering a computer representation of one or more values of the plurality of evaluation parameters on a user interface. The user interface may include a filter-based user interface (UI) including user-selectable filter fields for selecting at least one or more care groups from the plurality of care groups. In examples in which the wearer is associated, e.g. resides in, a care system that belongs to a group including two or more care systems, the filter-based UI may further include user-selectable filter fields for selecting one or more care systems from one or more care systems. The filter-based UI may be instructed to be rendered on the computer device through which the assessment tool is accessed, or on another computer device. One or more computer devices associated with a care system may render the filter-based UI, also referred to as an evaluation results UI, in response to the corresponding instructions from the evaluation server.

In examples, the evaluation server may instruct rendering the generated values of the plurality of evaluation parameters on the filter-based UI 122 of FIG. 1. In the method 500 illustrated in connection with FIG. 5, the values of the plurality of evaluation parameters may indicate a comparison between the first plurality of evaluation parameters and the second plurality of evaluation parameters. Accordingly, the filter-based UI may be rendered that allows receiving an indication of a selection of one or more profile groups, one or more care systems, one or more wards or another unit(s) of the care system, and one or more evaluation parameters, so that, responsive to the selection, a computer representation is generated on the filter-based UI in the form of a comparison between the first plurality of evaluation parameters and the second plurality of evaluation parameters, as discussed throughout the present disclosure.

In some embodiments, the filter-based user interface is configured to present, in response to receiving user input via the user-selectable filter fields, a computer representation of one or more values of the plurality of evaluation parameters corresponding to the selected one or more profile groups, and, in some examples, additionally one or more care systems. In some embodiments, the computer representation of the one or more values of the plurality of evaluation parameters includes a first icon representing a corresponding evaluation parameter from the plurality of evaluation parameters and a second icon related to the first icon and representing a value of the corresponding evaluation parameter.

The various methods or processes outlined in the present disclosure may be implemented in any suitable hardware. Additionally, the various methods or processes outlined herein may be implemented in a combination of hardware and of software executable on one or more processors that employ any one of a variety of operating systems or platforms. Any suitable combination of hardware and software may be employed to implement any of the examples discussed herein.

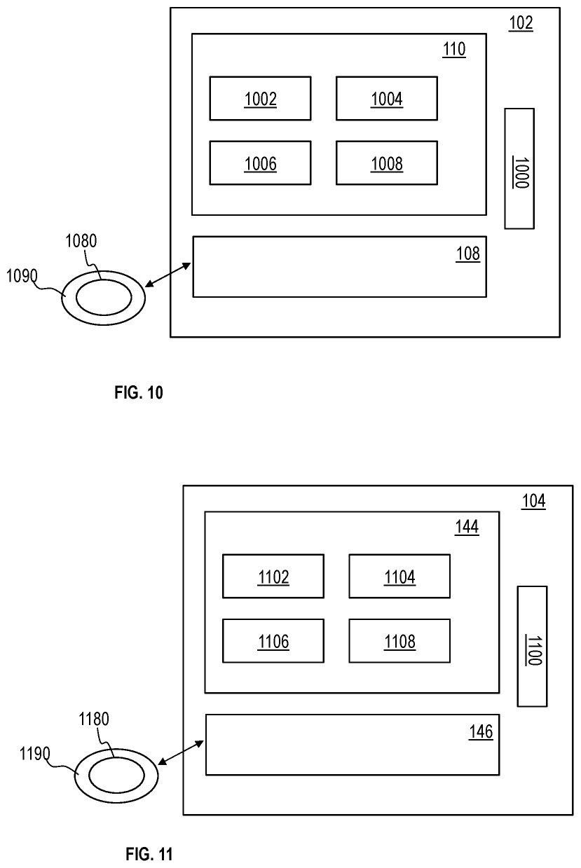

FIG. 10 is a schematic diagram of a computer system or device for implementing examples disclosed herein such as, e.g., computer device 102 shown in FIG. 1. The computer device 102 may include any collection of devices that individually or jointly execute a set or multiple sets of instructions to perform any one or more of the methods, processes, steps, or acts described herein. The computer device 102 may be a single multi-purpose programmable digital computing device, or a coordinated system of two or more multi-purpose computing device sharing processing power and jointly carrying out the techniques described herein, or a single computing device or coordinated system of computing device (co-located or geographically distributed) dedicated to executing the techniques described herein, one or more Field-Programmable Gate Arrays (FPGAs) for carrying out the techniques described herein, or any other suitable system or device.

The computer device 102 may include a processing circuitry such as at least one processor or processor device 110 and the memory 108. The computer device 102 also includes and an input and output interface 1000 configured to communicate with any devices, systems and/or components of examples in accordance with the present disclosure. The input and output interface 1000 may include a wireless and/or wired receiver, a wireless and/or wired transmitter, and/or a wireless and/or wired transceiver.

The processor device 110 may be a computing device that is programmed to execute instructions when the instructions are stored in a manner accessible to the computing device, such as in a data store, e.g., an on-chip cache or instruction register, a computer-readable storage medium accessible via a bus, a computer-readable storage medium accessible via one or more networks and accessible by the device/processor, etc. The processor device 110 may, for example, include a general-purpose processor, an application specific processor, a Digital Signal Processor (DSP), an Application Specific Integrated Circuit (ASIC), a Field Programmable Gate Array (FPGA), circuitry including processing components, a group of distributed processing components, a group of distributed computers configured for processing, or other programmable logic device, discrete gate or transistor logic, discrete hardware components, or any combination thereof designed to perform the functions described herein.

The memory 108 may be one or more devices for storing data and/or computer-executable instructions for executing or facilitating one or more processes described herein. The computer-executable instructions may be implemented as software, including as application software, system software, firmware, middleware, embedded code, or any other suitable type of computer code.

The memory 108 may include database components, object code components, script components, or other types of information structure for supporting the various methods or processes described herein. Any distributed or local memory device may be utilized with the systems and methods of this description. The memory 108 may be communicably connected to the processor device 110 e.g., via a circuit or any other wired, wireless, or network connection. The memory 108 may include non-volatile memory (e.g., read-only memory (ROM), erasable programmable read-only memory (EPROM), electrically erasable programmable read-only memory (EEPROM), etc.), and volatile memory (e.g., random-access memory (RAM)), or any other medium which can be used to carry or store desired program code in the form of machine-executable instructions or data structures and which can be accessed by a computer or other machine with the processor device 110.

The methods according to aspects of the present disclosure may be implemented by e.g. a computer program product 1080 or a computer program, including computer-executable instructions, i.e., software code portions, which, when executed on at least one processor, e.g., the processor device 110, cause the at least one processor to carry out the actions described herein, as performed by the computer device 102. In some examples, the computer program product 1080 is stored on a computer-readable storage medium 1090. The computer-readable storage medium 1090 may be, e.g., a disc, a universal serial bus (USB) stick, or any other type of computer-readable storage media. The computer-readable storage medium 1090, having stored thereon the computer program product, may include the instructions which, when executed on at least one processor, e.g., the processor device 110, cause the at least one processor to carry out the actions of the method described herein, as performed by the computer device 102.

As shown in FIG. 10, the computer device 102 may include a receiving unit 1002. The computer device 102, the processor device 110, and/or the receiving unit 1002 may be configured to, for each wearer from a plurality of wearers in a care system, receive, during an assessment time period, assessment time series data regarding a second process of use of a second set of one or more incontinence products worn by the wearer. The assessment time series data includes assessment values of assessment parameters related to the use of the second set of one or more incontinence products worn by the wearer; the second process of use of the second set of one or more incontinence products includes a plurality of second steps; and, for each profile group from a plurality of profile groups, a step from the plurality of second steps is associated with an estimated duration of time required to perform the step.

The computer device 102, the processor device 110, and/or the receiving unit 1002 may further be configured to receive, from the evaluation server, a recommendation for the care system regarding the second process of use of the second set of one or more incontinence products.

The computer device 102, the processor device 110, and/or the receiving unit 1002 may further be configured to receive, from the evaluation server, an indication to the care system, the indication recommending that the care system uses incontinence products of the second set.

The computer device 102 may include an acquiring unit 1004. The computer device 102, the processor device 110, and/or the acquiring unit 1004 may be configured to acquire, from a data storage device, a wearer profile record stored in the data storage device, the wearer profile record being created for the wearer, the wearer profile record indicating a profile group from the plurality of profile groups to which the wearer is assigned and metadata related to the wearer.

The computer device 102 may include a sending unit 1006. The computer device 102, the processor device 110, and/or the sending unit 1006 may be configured to send the wearer profile record to an evaluation server. The computer device 102, the processor device 110, and/or the sending unit 1006 may be configured to send the assessment time series data to the evaluation server. The computer device 102, the processor device 110, and/or the sending unit 1006 may be configured to, when a value of at least one of the plurality of evaluation parameters has reached or exceeded a threshold, automatically transmitting, from the computer device or from the evaluation server, to an external computer, a request for a certain amount of incontinence products of the second set to be delivered to the care system.

The computer device 102, the processor device 110, and/or the receiving unit 1002 may be configured to receive values of a plurality of evaluation parameters related to computer-generated evaluation of the second process of use of the second set of one or more incontinence products worn by the plurality of wearers, based on processing by the evaluation server of the assessment time series data and the wearer profile record for each wearer from the plurality of wearers. The values of the plurality of evaluation parameters may be received from the evaluation server.

As further shown in FIG. 10, the computer device 102 may include a generating unit 1008. The computer device 102, the processor device 110, and/or the generating unit 1008 may be configured to generate a computer representation of one or more values of the plurality of evaluation parameters corresponding to selected one or more profile groups for the care system from the one or more care systems. The computer representation may be generated on a UI such as e.g. a filter-based user UI. The a filter-based user UI may include user-selectable filter fields for selecting the one or more profile groups from the plurality of profile groups, and, in some examples, user-selectable filter fields for selecting one or more care systems from the one or more care systems. The filter-based UI may be configured to present, in response to receiving user input via the user-selectable filter fields, the computer representation of the one or more values of the plurality of evaluation parameters corresponding to the selected one or more profile groups and to the selected one or more care systems. The user interface may be rendered by an assessment tool.

FIG. 11 is a schematic diagram of a computer system or server for implementing examples disclosed herein such as, e.g., evaluation server 104 shown in FIG. 1. The evaluation server 104 may include any collection of servers or devices that individually or jointly execute a set or multiple sets of instructions to perform any one or more of the methods, processes, steps, or acts described herein. The evaluation server 104 may be a single multi-purpose programmable digital computing device, or a coordinated system of two or more multi-purpose computing device sharing processing power and jointly carrying out the techniques described herein, or a single computing device or coordinated system of computing device (co-located or geographically distributed) dedicated to executing the techniques described herein, one or more Field-Programmable Gate Arrays (FPGAs) for carrying out the techniques described herein, or any other suitable system or device.

The evaluation server 104 may include a processing circuitry such as at least one processor or processor device 144 and the memory 146. The evaluation server 104 also includes and an input and output interface 1100 configured to communicate with any devices, systems and/or components of examples in accordance with the present disclosure. The input and output interface 1100 may include a wireless and/or wired receiver, a wireless and/or wired transmitter, and/or a wireless and/or wired transceiver.

The processor device 144 may be a computing device that is programmed to execute instructions when the instructions are stored in a manner accessible to the computing device, such as in a data store, e.g., an on-chip cache or instruction register, a computer-readable storage medium accessible via a bus, a computer-readable storage medium accessible via one or more networks and accessible by the device/processor, etc., The processor device 144 may, for example, include a general-purpose processor, an application specific processor, a Digital Signal Processor (DSP), an Application Specific Integrated Circuit (ASIC), a Field Programmable Gate Array (FPGA), circuitry including processing components, a group of distributed processing components, a group of distributed computers configured for processing, or other programmable logic device, discrete gate or transistor logic, discrete hardware components, or any combination thereof designed to perform the functions described herein.

The memory 146 may be one or more devices for storing data and/or computer-executable instructions for executing or facilitating one or more processes described herein. The computer-executable instructions may be implemented as software, including as application software, system software, firmware, middleware, embedded code, or any other suitable type of computer code.

The memory 146 may include database components, object code components, script components, or other types of information structure for supporting the various methods or processes described herein. Any distributed or local memory device may be utilized with the systems and methods of this description. The memory 146 may be communicably connected to the processor device 144 e.g., via a circuit or any other wired, wireless, or network connection. The memory 146 may include non-volatile memory (e.g., read-only memory (ROM), erasable programmable read-only memory (EPROM), electrically erasable programmable read-only memory (EEPROM), etc.), and volatile memory (e.g., random-access memory (RAM)), or any other medium which can be used to carry or store desired program code in the form of machine-executable instructions or data structures and which can be accessed by a computer or other machine with the processor device 144.

The methods according to aspects of the present disclosure may be implemented by e.g. a computer program product 1180 or a computer program, including computer-executable instructions, i.e., software code portions, which, when executed on at least one processor, e.g., the processor device 144, cause the at least one processor to carry out the actions described herein, as performed by the evaluation server 104. In some examples, the computer program product 1180 is stored on a computer-readable storage medium 1190. The computer-readable storage medium 1190 may be, e.g., a disc, a universal serial bus (USB) stick, or any other type of computer-readable storage media. The computer-readable storage medium 1190, having stored thereon the computer program product, may include the instructions which, when executed on at least one processor, e.g., the processor device 144, cause the at least one processor to carry out the actions of the method described herein, as performed by the evaluation server 104.

As shown in FIG. 11, the evaluation server 104 may include a generating unit 1102. The evaluation server 104, the processor device 144, and/or the generating unit 1102 may be configured to generate or determine or compute values of a plurality of evaluation parameters related to computer-generated evaluation of the second process of use of the second set of one or more incontinence products worn by the plurality of wearers, based on processing of the assessment time series data and the wearer profile record for each wearer from the plurality of wearers. The values of the plurality of evaluation parameters may be generated as described above herein.

The evaluation server 104 may include a receiving unit 1104. The evaluation server 104, the processor device 144, and/or the receiving unit 1104 may be configured to receive assessment time series data regarding a second process of use of a second set of one or more incontinence products worn by the wearer. The assessment time series data may include assessment values of assessment parameters related to the use of the second set of one or more incontinence products worn by the wearer; the second process of use of the second set of one or more incontinence products may include a plurality of second steps; and, for each profile group from a plurality of profile groups, a step from the plurality of second steps may be associated with an estimated duration of time required to perform the step.

The evaluation server 104 may include an acquiring unit 1106. The evaluation server 104, the processor device 144, and/or the acquiring unit 1106 may be configured to acquire a wearer profile record, the wearer profile record being created for the wearer, and the wearer profile record indicating a profile group from the plurality of profile groups to which the wearer is assigned and metadata related to the wearer. The evaluation server 104, the processor device 144, and/or the acquiring unit 1106 may be configured to acquire other information, e.g., information from a product data storage, care system settings, evaluation settings, etc.

The evaluation server 104 may include a generating unit 1108. The evaluation server 104, the processor device 144, and/or the generating unit 1108 may be configured to generate a computer representation of one or more values of the plurality of evaluation parameters corresponding to selected one or more profile groups for the care system from the one or more care systems. The computer representation may be instructed to be generated or rendered on a computer device, e.g., on a user interface of computer device 102 or another computer device or system.

The computer device 102 and evaluation server 104 may include other units that implement the functionality of examples of the present disclosure

The operational steps described in any of the exemplary aspects herein are described to provide examples and discussion. The steps may be performed by hardware components, may be embodied in machine-executable instructions to cause a processor to perform the steps, or may be performed by a combination of hardware and software. Although a specific order of method steps may be shown or described, the order of the steps may differ. In addition, two or more steps may be performed concurrently or with partial concurrence.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Various inventive concepts may be embodied as one or more methods, of which examples have been provided. The acts performed as part of any method described herein may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms. Unless otherwise defined, all terms (including technical and scientific terms) used herein have the same meaning as commonly understood by one of ordinary skill in the art to which this disclosure belongs. It is further understood that terms used herein should be interpreted as having a meaning consistent with their meaning in the context of this specification and the relevant art and will not be interpreted in an idealized or overly formal sense unless expressly so defined herein.

It will be understood that, although the terms first, second, etc., may be used herein to describe various elements, these elements should not be limited by these terms. These terms are only used to distinguish one element from another. For example, a first element could be termed a second element, and, similarly, a second element could be termed a first element without departing from the scope of the present disclosure.

The indefinite articles “a” and “an,” as used herein, unless clearly indicated to the contrary, should be understood to mean “at least one.”

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phraseology and terminology used herein is for the purpose of description and should not be regarded as limiting. The use of “including,” “comprising,” “having,” “containing”, “involving”, and variations thereof, is meant to encompass the items listed thereafter and additional items.

Any embodiment or example disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

It is to be understood that the present disclosure is not limited to the aspects described above and illustrated in the drawings; rather, the skilled person will recognize that many changes and modifications may be made within the scope of the present disclosure and appended claims. In the drawings and specification, there have been disclosed aspects for purposes of illustration only and not for purposes of limitation, the scope of the inventive concepts being set forth in the following claims.

1. A computer-implemented method that is performed by at least one processor of an evaluation server configured to:
for each wearer from a plurality of wearers in a care system:
receiving, during a first assessment time period, first assessment time series data regarding a first process of use of a first set of one or more incontinence products worn by the wearer;
receiving, during a second assessment time period, second assessment time series data regarding a second process of use of a second set of one or more incontinent products worn the wearer; and
receiving a wearer profile record being created for the wearer, the wearer profile record indicating a profile group from the plurality of profile groups to which the wearer is assigned and metadata related to the wearer, wherein:
the first assessment time series data comprises assessment values of first assessment parameters related to the use of the first set of one or more incontinence products by the wearer, and the second assessment time series data comprises assessment values of second assessment parameters related to the use of the second set of incontinence products by the wearer;
the first process of use includes a plurality of first steps and the second process of use includes a plurality of second steps; and
for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generating values of a plurality of evaluation parameters indicating a comparison of the first process of use and the second process of use, based on processing the first assessment time series data, the second assessment time series data, and the wearer profile record for each wearer from the plurality of wearers.

2. The method of claim 1, wherein the values of the plurality of evaluation parameters indicate a comparison between a first plurality of evaluation parameters related to computer-generated evaluation of the first process of use of the first set, and a second plurality of evaluation parameters related to computer-generated evaluation of the second process of use of the second set.

3. The method of claim 2, wherein the values of the plurality of evaluation parameters indicate a ratio between the first plurality of evaluation parameters and the second plurality of evaluation parameters.

4. The method of claim 1, further comprising
after receiving the first assessment time series data, receiving sensor data acquired from a test incontinence product, the test incontinence product being used by the wearer during an information collecting time period, and the test incontinence product comprising a wireless sensor configured to measure voiding activity of the wearer using the test incontinence product as the sensor data comprising measured test voiding data, and configured to send the measured test voiding data to the evaluation server.

5. The method of claim 4, wherein the second set of one or more incontinent products is selected based on the sensor data acquired from the test incontinence product.

6. The method of claim 1, wherein the first assessment time series data and the second assessment time series data are received via an assessment user interface of an assessment tool.

7. The method of claim 1, wherein the first assessment time period and the second assessment time period have the same duration of time.

8. The method of claim 1, further comprising
instructing rendering a computer representation of one or more values of the plurality of evaluation parameters on a user interface.

9. The method of claim 8, wherein the user interface comprises a filter-based user interface comprising user-selectable filter fields for selecting at least one or more profile groups from the plurality of profile groups.

10. The method of claim 9, wherein the filter-based user interface is configured to present, in response to receiving user input via the user-selectable filter fields, a computer representation of one or more values of the plurality of evaluation parameters corresponding to the selected one or more profile groups.

11. The method of claim 10, wherein the computer representation of the one or more values of the plurality of evaluation parameters comprises a first icon representing a corresponding evaluation parameter from the plurality of evaluation parameters and a second icon related to the first icon and representing a value of the corresponding evaluation parameter.

12. The method of claim 1, wherein the wearer is assigned to the profile group from the plurality of profile groups based at least on a voiding pattern of the wearer, mobility of the wearer, and a cognitive ability of the wearer.

13. The method of claim 1, wherein the first assessment time series data comprises one or more out of skin assessment, a change of an incontinence product of the first set, feces in the incontinence product of the first set, a toilet visit, a cleansing and protection event, an occurrence of urine leakage, a clothes change event, a linen change event, an application of a day type of the incontinence product of the first set, and an application of a night type of the incontinence product of the first set.

14. The method of claim 1, wherein the second assessment time series data comprises one or more out of skin assessment, a change of the incontinence product of the second set, feces in the incontinence product of the second set, a toilet visit, a cleansing and protection event, an occurrence of urine leakage, a clothes change event, a linen change event, an application of a day type of the incontinence product of the second set, and an application of a night type of the incontinence product of the second set.

15. The method of claim 1, wherein the first assessment parameters related to the use of the first set of one or more incontinence products by the wearer and the second assessment parameters related to the use of the second set of one or more incontinence products by the wearer are same parameters.

16. The method of claim 1, further comprising:
when the value of at least one of the plurality of evaluation parameters has reached or exceeded a threshold, automatically transmitting, to an external computer, a request for a certain amount of incontinence products of the second set to be delivered to the care system.

17. The method of claim 1, further comprising automatically generating a recommendation for the care system regarding the second process of use of the second set of one or more incontinence products.

18. The method of claim 17, wherein the recommendation comprises a recommendation for a modification to at least one step from the plurality of second steps of the second process of use of the second set of one or more incontinence products.

19. The method of claim 17, wherein the recommendation comprises a recommendation for a modification to a configuration of an incontinence product of the second set.

20. The method of claim 1, further comprising generating an indication to the care system, the indication recommending that the care system uses incontinence products of the second set.

21. The method of claim 4, further comprising automatically generating a recommendation for the care system regarding the second process of use of the second set of one or more incontinence products.

22. The method of claim 21, wherein the recommendation comprises a recommendation for a modification to at least one step from the plurality of second steps of the second process of use of the second ser of one or more incontinence products.

23. The method of claim 21, wherein the recommendation comprises a recommendation for a modification to a configuration of an incontinence product of the second set.

24. The method of any one of claim 21, wherein the recommendation for the care system is generated using the sensor data acquired from the test incontinence product.

25. A computer program product comprising instructions, which, when executed on at least one processor, cause the at least one processor to carry out the method of claim 1.

26. A computer-readable storage medium having stored thereon a computer program product comprising instructions which, when executed on at least one processor, cause the at least one processor to carry out the method of claim 1.

27. An evaluation server comprising at least one processor that is configured to:
for each wearer from a plurality of wearers in a care system:
receive, during a first assessment time period, first assessment time series data regarding a first process of use of a first set of one or more incontinence products worn by the wearer;
receive, during a second assessment time period, second assessment time series data regarding a second process of use of a second set of one or more incontinent products worn the wearer; and
receive a wearer profile record being created for the wearer, the wearer profile record indicating a profile group from the plurality of profile groups to which the wearer is assigned and metadata related to the wearer, wherein:
the first assessment time series data comprises assessment values of first assessment parameters related to the use of the first set of one or more incontinence products by the wearer, and the second assessment time series data comprises assessment values of second assessment parameters related to the use of the second set of incontinence products by the wearer;
the first process of use includes a plurality of first steps and the second process of use includes a plurality of second steps; and
for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generate values of a plurality of evaluation parameters indicating a comparison of the first process of use and the second process of use, based on processing the first assessment time series data, the second assessment time series data, and the wearer profile record for each wearer from the plurality of wearers.

28. The evaluation server of claim 27, wherein the at least one processor is configured to perform a method wherein the values of the plurality of evaluation parameters indicate a comparison between a first plurality of evaluation parameters related to computer-generated evaluation of the first process of use of the first set, and a second plurality of evaluation parameters related to computer-generated evaluation of the second process of use of the second set.

29. The evaluation server of claim 27, wherein the first assessment time series data and the second assessment time series data are received from the computer device.

30. A system comprising a computer device and an evaluation server, the evaluation server comprising at least one processor that is configured to:
for each wearer from a plurality of wearers in a care system:
receive, during a first assessment time period, first assessment time series data regarding a first process of use of a first set of one or more incontinence products worn by the wearer;
receive, during a second assessment time period, second assessment time series data regarding a second process of use of a second set of one or more incontinent products worn the wearer;
receive a wearer profile record being created for the wearer, the wearer profile record indicating a profile group from the plurality of profile groups to which the wearer is assigned and metadata related to the wearer, wherein:
the first assessment time series data comprises assessment values of first assessment parameters related to the use of the first set of one or more incontinence products by the wearer, and the second assessment time series data comprises assessment values of second assessment parameters related to the use of the second set of incontinence products by the wearer;
the first process of use includes a plurality of first steps and the second process of use includes a plurality of second steps; and
for each profile group from a plurality of profile groups, a step from the plurality of first and second steps is associated with an estimated duration of time required to perform the step; and
in response to receiving the first and the second assessment time series data and receiving the wearer profile record for each wearer from the plurality of wearers, generate values of a plurality of evaluation parameters indicating a comparison of the first process of use and the second process of use, based on processing the first assessment time series data, the second assessment time series data, and the wearer profile record for each wearer from the plurality of wearers.

31. The system of claim 30, wherein the at least one processor of the evaluation server is configured to perform a method wherein the values of the plurality of evaluation parameters indicate a comparison between a first plurality of evaluation parameters related to computer-generated evaluation of the first process of use of the first set, and a second plurality of evaluation parameters related to computer-generated evaluation of the second process of use of the second set.
